(19) United States
(12) Patent Application Publication
(10) Pub. No.: US 2024/0148617 A1
(43) Pub. Date: May 9, 2024

(54) COATED CORE-SHELL MICROCAPSULES

(71) Applicant: FIRMENICH SA, Satigny (CH)

(72) Inventors: Valentina VALMACCO, Satigny (CH); Damien BERTHIER, Satigny (CH); Nicolas PARET, Satigny (CH); Christophe ROMER, Satigny (CH)

(21) Appl. No.: 18/550,549

(22) PCT Filed: Mar. 28, 2022
(86) PCT No.: PCT/EP22/58044

§ 371 Date: Sep. 14, 2023

(30) Foreign Application Priority Data
Mar. 31, 2021 (EP) 21166229.1

(51) Int. Cl. A61K 8/11; A61K 8/04; A61K 8/73; A61K 8/92; A61Q 5/02; A61Q 5/12; A61Q 13/00; A61Q 15/00; A61Q 19/10; B01J 13/16; B01J 13/22; C11D 3/00; C11D 3/22; C11D 3/384; C11D 3/50

(52) U.S. Cl. CPC A61K 8/11; A61K 8/044; A61K 8/736; A61K 8/737; A61K 8/925; A61Q 5/02; A61Q 5/12; A61Q 13/00; A61Q 15/00; A61Q 19/10; B01J 13/16; B01J 13/22; C11D 3/0015; C11D 3/222; C11D 3/384; C11D 3/505

(57) ABSTRACT

Disclosed herein is a core-shell microcapsule slurry including at least one core-shell microcapsule. The at least one core-shell microcapsule includes an oil-based core including a hydrophobic material, a polymeric shell and a coating having a first deposition aid, and a second deposition aid. Further disclosed herein are a perfumed consumer product and a method for preparing the same.

### TECHNICAL FIELD OF THE INVENTION

The present invention relates to a core-shell microcapsule slurry comprising at least one core-shell microcapsule, wherein the at least one core-shell microcapsule comprises an oil-based core comprising a hydrophobic material, a polymeric shell and a coating comprising a first deposition aid, and a second deposition aid, a perfumed consumer product and a method for preparing the same.

### BACKGROUND OF THE INVENTION

One of the problems faced by the perfumery industry lies in the relatively rapid loss of olfactive benefit provided by odoriferous compounds due to their high volatility, particularly that of “top-notes”. In order to tailor the release rates of volatiles, delivery system such as microcapsules containing active ingredients, for example a perfume, are needed to protect and later release the core payload when triggered. A key requirement from the industry regarding these systems is to survive suspension in challenging bases without physically dissociating or degrading. This is referred to as chemical stability of a delivery system. For instance, fragrance personal and household cleansers containing high levels of aggressive surfactant detergents are very challenging for the stability of delivery systems, such as microcapsules. High levels of surfactants also increase the speed of diffusion of actives out of the delivery system, such as a microcapsule. This leads to leakage of the actives during storage and a reduced impact when the microcapsules are triggered to release.

Another problem faced by the perfumery industry lies in the provision of capsule performance which is controlled by their storage stability, shell breakage to release perfume core and deposition on targeted substrate for the treatment of which the end product is intended to be used, such as textile, skin, hair or other surfaces, so as to possibly remain on the substrate even after a rinsing step. The deposition is usually controlled by the introduction of conventional deposition aid partners such as synthetic cationic copolymer of acrylamide and derivatives. Preparation of deposition aid partners with other functionalities such as alkyl chains and functional groups is very challenging.

There is a need in the industry for improving the ability of delivery systems to deposit on a substrate and to adhere on the substrate, while performing in terms of release and stability.

Moreover, in addition to the performance in terms of stability, deposition and olfactive performance, the consumer demand for eco-friendly delivery systems is more and more important and is driving the development of new delivery systems.

There is therefore still a need to provide new microcapsules using more eco-friendly materials, while not compromising on the performance of the microcapsules.

The present invention satisfies these and other needs of the industry.

### DETAILED DESCRIPTION OF THE INVENTION

Unless stated otherwise, percentages (%) are meant to designate percent by weight of a composition.

By “hydrophobic material”, it is meant a material which forms a two-phase dispersion when mixed with water. According to the invention, the hydrophobic material can be “inert” material like solvents or active ingredients. According to an embodiment, the hydrophobic material is a hydrophobic active ingredient.

By “active ingredient”, it is meant a single compound or a combination of ingredients.

By “perfume oil”, it is meant a single perfuming or a mixture of several perfuming compounds.

By “consumer product” or “end-product” it is meant a manufactured product ready to be distributed, sold and used by a consumer.

A “microcapsule”, or the similar, in the present invention has a morphology that can vary from a core-shell to a matrix type. According to one embodiment, it is of the core-shell type. In this case, the microcapsules comprise a core based on a hydrophobic material, typically a perfume, and a polymeric shell surrounding the oil core.

Microcapsules have a microcapsule size distribution in the micron range (e.g. a mean diameter) comprised between about 1 and 3000 microns, preferably comprised between 1 and 1000 microns, more preferably between 1 and 500 microns, and even more preferably between 5 and 50 microns.

By “particle size” it is meant an average diameter of particles based on size distribution measured by dynamic light scattering (DLS) using Zetasizer Nano ZS equipment from Malvern Instruments Ltd., UK when particles are dispersed into a water phase.

By “microcapsules size” it is meant the volume mean diameter (D[4,3]) of the relevant capsules, capsules suspension as obtained by laser light scattering of a diluted sample in a Malvern Mastersizer 3000.

By “microcapsule slurry”, it is meant microcapsule(s) that is (are) dispersed in a liquid. According to an embodiment, the slurry is an aqueous slurry, i.e the microcapsule(s) is (are) dispersed in an aqueous phase.

The present invention relates to a core-shell microcapsule slurry comprising
at least one core-shell microcapsule, wherein the at least one core-shell microcapsule comprises
   an oil-based core comprising a hydrophobic material, preferably a perfume,
   a polymeric shell, and
   a coating comprising a first deposition aid, and
a second deposition aid,

wherein the first and second deposition aid are different deposition aids.

For the sake of clarity, by the expression core-shell microcapsule it is understood that the hydrophobic material in the oil-based core is surrounded by the shell of the microcapsule. “Shell” and “wall” are used indifferently in the present invention.

According to the present invention, the core-shell microcapsule comprises an oil-based core comprising a hydrophobic material.

By “oil” it is understood an organic phase that is liquid at about 20° C. which forms the core of the core-shell microcapsules.

### Hydrophobic Material

The hydrophobic material according to the invention can be “inert” material like solvents or active ingredients.

When hydrophobic materials are active ingredients, they are preferably chosen from the group consisting of flavors, flavor ingredients, perfumes, perfume ingredients, nutraceuticals, cosmetics, pest control agents, biocide actives and mixtures thereof.

According to a particular embodiment, the hydrophobic material comprises a mixture of a perfume with another ingredient selected from the group consisting of nutraceuticals, cosmetics, pest control agents and biocide actives.

According to a particular embodiment, the hydrophobic material comprises a mixture of biocide actives with another ingredient selected from the group consisting of perfumes, nutraceuticals, cosmetics, pest control agents.

According to a particular embodiment, the hydrophobic material comprises a mixture of pest control agents with another ingredient selected from the group consisting of perfumes, nutraceuticals, cosmetics, biocide actives.

According to a particular embodiment, the hydrophobic material comprises a perfume.

According to a particular embodiment, the hydrophobic material consists of a perfume.

According to a particular embodiment, the hydrophobic material consists of biocide actives.

According to a particular embodiment, the hydrophobic material consists of pest control agents.

By “perfume” (or also “perfume oil”) what is meant here is an ingredient or a composition that is a liquid at about 20° C. According to any one of the above embodiments said perfume oil can be a perfuming ingredient alone or a mixture of ingredients in the form of a perfuming composition. As a “perfuming ingredient” it is meant here a compound, which is used for the primary purpose of conferring or modulating an odor. In other words such an ingredient, to be considered as being a perfuming one, must be recognized by a person skilled in the art as being able to at least impart or modify in a positive or pleasant way the odor of a composition, and not just as having an odor. For the purpose of the present invention, perfume oil also includes a combination of perfuming ingredients with substances which together improve, enhance or modify the delivery of the perfuming ingredients, such as perfume precursors, emulsions or dispersions, as well as combinations which impart an additional benefit beyond that of modifying or imparting an odor, such as long-lastingness, blooming, malodor counteraction, antimicrobial effect, microbial stability, pest control.

The nature and type of the perfuming ingredients present in the oil phase do not warrant a more detailed description here, which in any case would not be exhaustive, the skilled person being able to select them on the basis of its general knowledge and according to intended use or application and the desired organoleptic effect. In general terms, these perfuming ingredients belong to chemical classes as varied as alcohols, aldehydes, ketones, esters, ethers, acetates, nitriles, terpenoids, nitrogenous or sulfurous heterocyclic compounds and essential oils, and said perfuming co-ingredients can be of natural or synthetic origin. Many of these co-ingredients are in any case listed in reference texts such as the book by S. Arctander, Perfume and Flavor Chemicals, 1969, Montclair, New Jersey, USA, or its more recent versions, or in other works of a similar nature, as well as in the abundant patent literature in the field of perfumery.

In particular one may cite perfuming ingredients which are commonly used in perfume formulations, such as:
Aldehydic ingredients: decanal, dodecanal, 2-methyl-undecanal, 10-undecenal, octanal, nonanal and/or nonenal;
Aromatic-herbal ingredients: eucalyptus oil, camphor, eucalyptol, 5-methyltricyclo[6.2.1.0˜2,7˜]undecan-4-one, 1-methoxy-3-hexanethiol, 2-ethyl-4,4-dimethyl-1,3-oxathiane, 2,2,7/8,9/10-Tetramethylspiro[5.5]undec-8-en-1-one, menthol and/or alpha-pinene;
Balsamic ingredients: coumarin, ethylvanillin and/or vanillin;
Citrus ingredients: dihydromyrcenol, citral, orange oil, linalyl acetate, citronellyl nitrile, orange terpenes, limonene, 1-p-menthen-8-yl acetate and/or 1,4(8)-p-menthadiene;
Floral ingredients: methyl dihydrojasmonate, linalool, citronellol, phenylethanol, 3-(4-tert-butylphenyl)-2-methylpropanal, hexylcinnamic aldehyde, benzyl acetate, benzyl salicylate, tetrahydro-2-isobutyl-4-methyl-4(2H)-pyranol, beta ionone, methyl 2-(methylamino)benzoate, (E)-3-methyl-4-(2,6,6-trimethyl-2-cyclohexen-1-yl)-3-buten-2-one, (1E)-1-(2,6,6-trimethyl-2-cyclohexen-1-yl)-1-penten-3-one, 1-(2,6,6-trimethyl-1,3-cyclohexadien-1-yl)-2-buten-1-one, (2E)-1-(2,6,6-trimethyl-2-cyclohexen-1-yl)-2-buten-1-one, (2E)-1-[2,6,6-trimethyl-3-cyclohexen-1-yl]-2-buten-1-one, (2E)-1-(2,6,6-trimethyl-1-cyclohexen-1-yl)-2-buten-1-one, 2,5-dimethyl-2-indanmethanol, 2,6,6-trimethyl-3-cyclohexene-1-carboxylate, 3-(4,4-dimethyl-1-cyclohexen-1-yl)propanal, hexyl salicylate, 3,7-dimethyl-1,6-nonadien-3-ol, 3-(4-isopropylphenyl)-2-methylpropanal, verdyl acetate, geraniol, p-menth-1-en-8-ol, 4-(1,1-dimethylethyl)-1-cyclohexyle acetate, 1,1-dimethyl-2-phenylethyl acetate, 4-cyclohexyl-2-methyl-2-butanol, amyl salicylate, high cis methyl dihydrojasmonate, 3-methyl-5-phenyl-1-pentanol, verdyl proprionate, geranyl acetate, tetrahydro linalool, cis-7-p-menthanol, propyl (S)-2-(1,1-dimethylpropoxy)propanoate, 2-methoxynaphthalene, 2,2,2-trichloro-1-phenylethyl acetate, 4/3-(4-hydroxy-4-methylpentyl)-3-cyclohexene-1-carbaldehyde, amylcinnamic aldehyde, 8-decen-5-olide, 4-phenyl-2-butanone, isononyle acetate, 4-(1,1-dimethylethyl)-1-cyclohexyl acetate, verdyl isobutyrate and/or mixture of methylionones isomers;
Fruity ingredients: gamma-undecalactone, 2,2,5-trimethyl-5-pentylcyclopentanone, 2-methyl-4-propyl-1,3-oxathiane, 4-decanolide, ethyl 2-methyl-pentanoate, hexyl acetate, ethyl 2-methylbutanoate, gamma-nonalactone, allyl heptanoate, 2-phenoxyethyl isobutyrate, ethyl 2-methyl-1,3-dioxolane-2-acetate, 3-(3,3/1,1-dimethyl-5-indanyl)propanal, diethyl 1,4-cyclohexanedicarboxylate, 3-methyl-2-hexen-1-yl acetate, 1-[3,3-dimethylcyclohexyl]ethyl [3-ethyl-2-oxiranyl]acetate and/or diethyl 1,4-cyclohexane dicarboxylate;
Green ingredients: 2-methyl-3-hexanone (E)-oxime, 2,4-dimethyl-3-cyclohexene-1-carbaldehyde, 2-tert-butyl-1-cyclohexyl acetate, styrallyl acetate, allyl (2-methylbutoxy)acetate, 4-methyl-3-decen-5-ol, diphenyl ether, (Z)-3-hexen-1-ol and/or 1-(5,5-dimethyl-1-cyclohexen-1-yl)-4-penten-1-one;
Musk ingredients: 1,4-dioxa-5,17-cycloheptadecanedione, (Z)-4-cyclopentadecen-1-one, 3-methylcyclopentadecanone, 1-oxa-12-cyclohexadecen-2-one, 1-oxa-13-cyclohexadecen-2-one, (9Z)-9-cycloheptadecen-1-one, 2-{1S)-1-[(1R)-3,3-dimethylcyclohexyl]ethoxy}-2-oxoethyl propionate 3-methyl-5-cyclopentadecen-1-one, 1,3,4,6,7,8-hexahydro-4,6,6,7,8,8-hexamethyl-cyclopenta-g-2-benzopyrane, (1S,1′R)-2-[1-(3′,3′-dimethyl-1′-cyclohexyl)ethoxy]-2-methylpropyl propanoate, oxacyclohexadecan-2-one and/or (1S,1′R)-[1-(3′,3′-dimethyl-1′-cyclohexyl)ethoxycarbonyl]methyl propanoate;
Woody ingredients: 1-[(1RS,6SR)-2,2,6-trimethylcyclohexyl]-3-hexanol, 3,3-dimethyl-5-[(1R)-2,2,3-trimethyl-3-cyclopenten-1-yl]-4-penten-2-ol, 3,4′-dimethylspiro[oxirane-2,9′-tricyclo[6.2.1.02,7]undec[4]ene, (1-ethoxyethoxy)cyclododecane, 2,2,9,11-tetramethylspiro[5.5]undec-8-en-1-yl acetate, 1-(octahydro-2,3,8,8-tetramethyl-2-naphtalenyl)-1-ethanone, patchouli oil, terpenes fractions of patchouli oil, Clearwood®, (1′R,E)-2-ethyl-4-(2′,2′,3′-trimethyl-3′-cyclopenten-1′-yl)-2-buten-1-ol, 2-ethyl-4-(2,2,3-trimethyl-3-cyclopenten-1-yl)-2-buten-1-ol, methyl cedryl ketone, 5-(2,2,3-trimethyl-3-cyclopentenyl)-3-methylpentan-2-ol, 1-(2,3,8,8-tetramethyl-1,2,3,4,6,7,8,8a-octahydronaphthalen-2-yl)ethan-1-one and/or isobornyl acetate;
Other ingredients (e.g. amber, powdery spicy or watery): dodecahydro-3a,6,6,9a-tetramethyl-naphtho[2,1-b]furan and any of its stereoisomers, heliotropin, anisic aldehyde, eugenol, cinnamic aldehyde, clove oil, 3-(1,3-benzodioxol-5-yl)-2-methylpropanal, 7-methyl-2H-1,5-benzodioxepin-3(4H)-one, 2,5,5-trimethyl-1,2,3,4,4a,5,6,7-octahydro-2-naphthalenol, 1-phenylvinyl acetate, 6-methyl-7-oxa-1-thia-4-azaspiro[4.4]nonan and/or 3-(3-isopropyl-1-phenyl)butanal.

According to a particular embodiment, the perfume or perfume formulation comprises a fragrance modulator (that can be used in addition to the hydrophobic solvent when present or as substitution of the hydrophobic solvent when there is no hydrophobic solvent).

Preferably, the fragrance modulator is defined as a fragrance material with
a vapor pressure of less than 0.0008 Torr at 22° C.;
a clogP of 3.5 and higher, preferably 4.0 and higher and more preferably 4.5
at least two Hansen solubility parameters selected from a first group consisting of: an atomic dispersion force from 12 to 20, a dipole moment from 1 to 7, and a hydrogen bonding from 2.5 to 11,
at least two Hansen solubility parameters selected from a second group consisting of: an atomic dispersion force from 14 to 20, a dipole moment from 1 to 8, and a hydrogen bonding from 4 to 11, when in solution with a compound having a vapor pressure range of 0.0008 to 0.08 Torr at 22° C.

Preferably as examples the following ingredients can be listed as fragrance modulators but the list in not limited to the following materials: alcohol C12, oxacyclohexadec-12/13-en-2-one, 3-[(2′,2′,3′-trimethyl-3′-cyclopenten-1′-yl)methoxy]-2-butanol, cyclohexadecanone, (Z)-4-cyclopentadecen-1-one, cyclopentadecanone, (8Z)-oxacycloheptadec-8-en-2-one, 2-[5-(tetrahydro-5-methyl-5-vinyl-2-furyl)-tetrahydro-5-methyl-2-furyl]-2-propanol, muguet aldehyde, 1,5,8-trimethyl-13-oxabicyclo[10.1.0]trideca-4,8-diene, (+−)-4,6,6,7,8,8-hexamethyl-1,3,4,6,7,8-hexahydrocyclopenta[g]isochromene, (+)-(1S,2S,3S,5R)-2,6,6-trimethylspiro[bicyclo[3.1.1]heptane-3,1′-cyclohexane]-2′-en-4′-one, oxacyclohexadecan-2-one, 2-{(1S)-1-[(1R)-3,3-dimethylcyclohexyl]ethoxy}-2-oxoethyl propionate, (+)-(4R,4aS,6R)-4,4a-dimethyl-6-(1-propen-2-yl)-4,4a,5,6,7,8-hexahydro-2(3H)-naphthalenone, amylcinnamic aldehyde, hexylcinnamic aldehyde, hexyl salicylate, (1E)-1-(2,6,6-trimethyl-1-cyclohexen-1-yl)-1,6-heptadien-3-one, (9Z)-9-cycloheptadecen-1-one.

It is also understood that said ingredients may also be compounds known to release in a controlled manner various types of perfuming compounds also known as properfume or profragrance. Non-limiting examples of suitable properfumes may include 4-(dodecylthio)-4-(2,6,6-trimethyl-2-cyclohexen-1-yl)-2-butanone, 4-(dodecylthio)-4-(2,6,6-trimethyl-1-cyclohexen-1-yl)-2-butanone, 3-(dodecylthio)-1-(2,6,6-trimethyl-3-cyclohexen-1-yl)-1-butanone, 2-(dodecylthio)octan-4-one, 2-phenylethyl oxo(phenyl)acetate, 3,7-dimethylocta-2,6-dien-1-yl oxo(phenyl)acetate, (Z)-hex-3-en-1-yl oxo(phenyl)acetate, 3,7-dimethyl-2,6-octadien-1-yl hexadecanoate, bis(3,7-dimethylocta-2,6-dien-1-yl) succinate, (2-((2-methylundec-1-en-1-yl)oxy)ethyl)benzene, 1-methoxy-4-(3-methyl-4-phenethoxybut-3-en-1-yl)benzene, (3-methyl-4-phenethoxybut-3-en-1-yl)benzene, 1-(((Z)-hex-3-en-1-yl)oxy)-2-methylundec-1-ene, (2-((2-methylundec-1-en-1-yl)oxy)ethoxy)benzene, 2-methyl-1-(octan-3-yloxy)undec-1-ene, 1-methoxy-4-(1-phenethoxyprop-1-en-2-yl)benzene, 1-methyl-4-(1-phenethoxyprop-1-en-2-yl)benzene, 2-(1-phenethoxyprop-1-en-2-yl)naphthalene, (2-phenethoxyvinyl)benzene, 2-(1-((3,7-dimethyloct-6-en-1-yl)oxy)prop-1-en-2-yl)naphthalene, (2-((2-pentylcyclopentylidene)methoxy)ethyl)benzene, 4-allyl-2-methoxy-1-((2-methoxy-2-phenylvinyl)oxy)benzene, (2-((2-heptylcyclopentylidene)methoxy)ethyl)benzene, 1-isopropyl-4-methyl-2-((2-pentylcyclopentylidene)methoxy)benzene, 2-methoxy-1-((2-pentylcyclopentylidene)methoxy)-4-propylbenzene, 3-methoxy-4-((2-methoxy-2-phenylvinyl)oxy)benzaldehyde, 4-((2-(hexyloxy)-2-phenylvinyl)oxy)-3-methoxybenzaldehyde or a mixture thereof.

The perfuming ingredients may be dissolved in a solvent of current use in the perfume industry. The solvent is preferably not an alcohol. Examples of such solvents are diethyl phthalate, isopropyl myristate, Abalyn® (rosin resins, available from Eastman), benzyl benzoate, ethyl citrate, limonene or other terpenes, or isoparaffins. Preferably, the solvent is very hydrophobic and highly sterically hindered, like for example Abalyn® or benzyl benzoate. Preferably the perfume comprises less than 30% of solvent. More preferably the perfume comprises less than 20% and even more preferably less than 10% of solvent, all these percentages being defined by weight relative to the total weight of the perfume. Most preferably, the perfume is essentially free of solvent.

According to a particular embodiment, the perfume comprises at least 35% of perfuming ingredients having a log P above 3.

Log P is the common logarithm of estimated octanol-water partition coefficient, which is known as a measure of lipophilicity.

The Log P values of many perfuming compound have been reported, for example, in the Pomona92 database, available from Daylight Chemical Information Systems, Inc. (Daylight CIS), Irvine, Calif., which also contains citations to the original literature. Log P values are most conveniently calculated by the “CLOGP” program, also available from Daylight CIS. This program also lists experimental log P values when they are available in the Pomona92 database. The “calculated log P” (c Log P) is determined by the fragment approach of Hansch and Leo (cf., A. Leo, in Comprehensive Medicinal Chemistry, Vol. 4, C. Hansch, P. G. Sammens, J. B. Taylor and C. A. Ramsden, Eds., p. 295, Pergamon Press, 1990). The fragment approach is based on the chemical structure of each perfume oil ingredient, and takes into account the numbers and types of atoms, the atom connectivity, and chemical bonding. The c Log P values, which are the most reliable and widely used estimates for this physicochemical property, are preferably used instead of the experimental Log P values in the selection of perfuming compounds which are useful in the present invention.

In a particular embodiment, the perfume oil comprises at least 40 wt. %, preferably at least 50 wt. %, more preferably at least 60 wt. % of ingredients having a log P above 3, preferably above 3.5 and even more preferably above 3.75.

Preferably, the perfume oil contains less than 10 wt. % of its own weight of primary alcohols, less than 15 wt. % of its own weight of secondary alcohols and less than 20% of its own weight of tertiary alcohols. Advantageously, the perfume used in the invention does not contain any primary alcohols and contains less than 15 wt. % of secondary and tertiary alcohols.

According to a particular embodiment, the perfume comprises at least 20 wt. %, preferably at least 25 wt. %, more preferably at least 40 wt. % of Bulky materials of groups 1 to 6, preferably 3 to 6.

The term Bulky materials is herein understood as perfuming ingredients having a high steric hindrance, i.e. having a substitution pattern which provides high steric hindrance and thus the Bulky materials are in particular those from one of the following groups:
Group 1: perfuming ingredients comprising a cyclohexane, cyclohexene, cyclohexanone or cyclohexenone ring substituted with at least one 1 to 4 nodes comprising substituent, preferably at least one linear or branched C1 to C4 alkyl or alkenyl substituent;
Group 2: perfuming ingredients comprising a cyclopentane, cyclopentene, cyclopentanone or cyclopentenone ring substituted with at least one 4 or more nodes comprising substituent, preferably at least one linear or branched C4 or longer, preferably C4 to C8 alkyl or alkenyl substituent;
Group 3: perfuming ingredients comprising a phenyl ring or perfuming ingredients comprising a cyclohexane, cyclohexene, cyclohexanone or cyclohexenone ring substituted with at least one 5 or more nodes comprising substituent, preferably at least one linear or branched C5 or longer, preferably C5 to C8, alkyl or alkenyl substituent, or with at least one phenyl substituent and optionally one or more 1 to 3 nodes comprising substituents, preferably one or more linear or branched C1 to C3 alkyl or alkenyl substituents;
Group 4: perfuming ingredients comprising at least two fused or linked 5 membered or 6 membered rings, preferably at least two fused or linked C5 and/or C6 rings;
Group 5: perfuming ingredients comprising a camphor-like ring structure, i.e. two 5 or 6 membered rings that are fused in a bridge-type fashion;
Group 6: perfuming ingredients comprising at least one 7 to 20 membered ring, preferably at least one C7 or C20 ring structure.

The term nodes as understood in this context means any atom which is able to provide at least two, preferably at least 3, more preferably 4, bonds to further atoms. Particular examples of nodes as herein understood are carbon atoms (up to 4 bonds to further atoms), nitrogen atoms (up to 3 bonds to further atoms), oxygen atoms (up to 2 bonds to further atoms) and sulfur (up to 2 bonds to further atoms). Particular examples of further atoms as understood in this context could be carbon atoms, nitrogen atoms, sulfur atoms, oxygen atoms and hydrogen atoms.

Examples of ingredients from each of these groups are:
Group 1: 2,4-dimethyl-3-cyclohexene-1-carbaldehyde (origin: Firmenich SA, Geneva, Switzerland), isocyclocitral, menthone, isomenthone, methyl 2,2-dimethyl-6-methylene-1-cyclohexanecarboxylate (origin: Firmenich SA, Geneva, Switzerland), nerone, terpineol, dihydroterpineol, terpenyl acetate, dihydroterpenyl acetate, dipentene, eucalyptol, hexylate, rose oxide, (S)-1,8-p-menthadiene-7-ol (origin: Firmenich SA, Geneva, Switzerland), 1-p-menthene-4-ol, (1RS,3RS,4SR)-3-p-mentanyl acetate, (1R,2S,4R)-4,6,6-trimethyl-bicyclo[3,1,1]heptan-2-ol, tetrahydro-4-methyl-2-phenyl-2H-pyran (origin: Firmenich SA, Geneva, Switzerland), cyclohexyl acetate, cyclanol acetate, 1,4-cyclohexane diethyldicarboxylate (origin: Firmenich SA, Geneva, Switzerland), (3ARS,6SR,7ASR)-perhydro-3,6-dimethyl-benzo[B]furan-2-one (origin: Firmenich SA, Geneva, Switzerland), ((6R)-perhydro-3,6-dimethyl-benzo[B]furan-2-one (origin: Firmenich SA, Geneva, Switzerland), 2,4,6-trimethyl-4-phenyl-1,3-dioxane, 2,4,6-trimethyl-3-cyclohexene-1-carbaldehyde;
Group 2: (E)-3-methyl-5-(2,2,3-trimethyl-3-cyclopenten-1-yl)-4-penten-2-ol (origin: Givaudan SA, Vernier, Switzerland), (1′R,E)-2-ethyl-4-(2′,2′,3′-trimethyl-3′-cyclopenten-1′-yl)-2-buten-1-ol (origin: Firmenich SA, Geneva, Switzerland), (1′R,E)-3,3-dimethyl-5-(2′,2′,3′-trimethyl-3′-cyclopenten-1′-yl)-4-penten-2-ol (origin: Firmenich SA, Geneva, Switzerland), 2-heptylcyclopentanone, methyl-cis-3-oxo-2-pentyl-1-cyclopentane acetate (origin: Firmenich SA, Geneva, Switzerland), 2,2,5-Trimethyl-5-pentyl-1-cyclopentanone (origin: Firmenich SA, Geneva, Switzerland), 3,3-dimethyl-5-(2,2,3-trimethyl-3-cyclopenten-1-yl)-4-penten-2-ol (origin: Firmenich SA, Geneva, Switzerland), 3-methyl-5-(2,2,3-trimethyl-3-cyclopenten-1-yl)-2-pentanol (origin, Givaudan SA, Vernier, Switzerland);
Group 3: damascones, 1-(5,5-dimethyl-1-cyclohexen-1-yl)-4-penten-1-one (origin: Firmenich SA, Geneva, Switzerland), nectalactone ((1′R)-2-[2-(4′-methyl-3′-cyclohexen-1′-yl)propyl]cyclopentanone), alpha-ionone, beta-ionone, damascenone, mixture of 1-(5,5-dimethyl-1-cyclohexen-1-yl)-4-penten-1-one and 1-(3,3-dimethyl-1-cyclohexen-1-yl)-4-penten-1-one (origin: Firmenich SA, Geneva, Switzerland), 1-(2,6,6-trimethyl-1-cyclohexen-1-yl)-2-buten-1-one (origin: Firmenich SA, Geneva, Switzerland), (1S,1′R)-[1-(3′,3′-Dimethyl-1′-cyclohexyl)ethoxycarbonyl]methyl propanoate (origin: Firmenich SA, Geneva, Switzerland), 2-tert-butyl-1-cyclohexyl acetate (origin: International Flavors and Fragrances, USA), 1-(2,2,3,6-tetramethyl-cyclohexyl)-3-hexanol (origin: Firmenich SA, Geneva, Switzerland), trans-1-(2,2,6-trimethyl-1-cyclohexyl)-3-hexanol (origin: Firmenich SA, Geneva, Switzerland), (E)-3-methyl-4-(2,6,6-trimethyl-2-cyclohexen-1-yl)-3-buten-2-one, terpenyl isobutyrate, 4-(1,1-dimethylethyl)-1-cyclohexyl acetate (origin: Firmenich SA, Geneva, Switzerland), 8-methoxy-1-p-menthene, (1S,1′R)-2-[1-(3′,3′-dimethyl-1′-cyclohexyl) ethoxy]-2-methylpropyl propanoate (origin: Firmenich SA, Geneva, Switzerland), para tert-butylcyclohexanone, menthenethiol, 1-methyl-4-(4-methyl-3-pentenyl)-3-cyclohexene-1-carbaldehyde, allyl cyclohexylpropionate, cyclohexyl salicylate, 2-methoxy-4-methylphenyl methyl carbonate, ethyl 2-methoxy-4-methylphenyl carbonate, 4-ethyl-2-methoxyphenyl methyl carbonate;
Group 4: Methyl cedryl ketone (origin: International Flavors and Fragrances, USA), a mixture of (1RS,2SR,6RS,7RS,8SR)-tricyclo[5.2.1.0˜2,6˜]dec-3-en-8-yl 2-methylpropanoate and (1RS,2SR,6RS,7RS,8SR)-tricyclo[5.2.1.0˜2,6˜]dec-4-en-8-yl 2-methylpropanoate, vetyverol, vetyverone, 1-(octahydro-2,3,8,8-tetramethyl-2-naphtalenyl)-1-ethanone (origin: International Flavors and Fragrances, USA), (5RS,9RS,10SR)-2,6,9,10-tetramethyl-1-oxaspiro[4.5]deca-3,6-diene and the (5RS,9SR,10RS) isomer, 6-ethyl-2,10,10-trimethyl-1-oxaspiro[4.5]deca-3,6-diene, 1,2,3,5,6,7-hexahydro-1,1,2,3,3-pentamethyl-4-indenone (origin: International Flavors and Fragrances, USA), a mixture of 3-(3,3-dimethyl-5-indanyl)propanal and 3-(1,1-dimethyl-5-indanyl)propanal (origin: Firmenich SA, Geneva, Switzerland), 3′,4-dimethyl-tricyclo[6.2.1.0(2,7)]undec-4-ene-9-spiro-2′-oxirane (origin: Firmenich SA, Geneva, Switzerland), 9/10-ethyldiene-3-oxatricyclo[6.2.1.0(2,7)]undecane, (perhydro-5,5,8A-trimethyl-2-naphthalenyl acetate (origin: Firmenich SA, Geneva, Switzerland), octalynol, (dodecahydro-3a,6,6,9a-tetramethyl-naphtho[2,1-b]furan, origin: Firmenich SA, Geneva, Switzerland), tricyclo[5.2.1.0(2,6)]dec-3-en-8-yl acetate and tricyclo[5.2.1.0(2,6)]dec-4-en-8-yl acetate as well as tricyclo[5.2.1.0(2,6)]dec-3-en-8-yl propanoate and tricyclo[5.2.1.0(2,6)]dec-4-en-8-yl propanoate, (+)-(1S,2S,3S)-2,6,6-trimethyl-bicyclo[3.1.1]heptane-3-spiro-2′-cyclohexen-4′-one;
Group 5: camphor, borneol, isobornyl acetate, 8-isopropyl-6-methyl-bicyclo[2.2.2]oct-5-ene-2-carbaldehyde, pinene, camphene, 8-methoxycedrane, (8-methoxy-2,6,6,8-tetramethyl-tricyclo[5.3.1.0(1,5)]undecane (origin: Firmenich SA, Geneva, Switzerland), cedrene, cedrenol, cedrol, mixture of 9-ethylidene-3-oxatricyclo[6.2.1.0(2,7)]undecan-4-one and 10-ethylidene-3-oxatricyclo[6.2.1.0(2,7)]undecan-4-one (origin: Firmenich SA, Geneva, Switzerland), 3-methoxy-7,7-dimethyl-10-methylene-bicyclo[4.3.1]decane (origin: Firmenich SA, Geneva, Switzerland);
Group 6: (trimethyl-13-oxabicyclo-[10.1.0]-trideca-4,8-diene (origin: Firmenich SA, Geneva, Switzerland), Ambrettolide LG ((E)-9-hexadecen-16-olide, origin: Firmenich SA, Geneva, Switzerland), pentadecenolide (origin: Firmenich SA, Geneva, Switzerland), muscenone (3-methyl-(4/5)-cyclopentadecenone, origin: Firmenich SA, Geneva, Switzerland), 3-methylcyclopentadecanone (origin: Firmenich SA, Geneva, Switzerland), pentadecanolide (origin: Firmenich SA, Geneva, Switzerland), cyclopentadecanone (origin: Firmenich SA, Geneva, Switzerland), 1-ethoxyethoxy)cyclododecane (origin: Firmenich SA, Geneva, Switzerland), 1,4-dioxacycloheptadecane-5,17-dione, 4,8-cyclododecadien-1-one;
Group 7: (+−)-2-methyl-3-[4-(2-methyl-2-propanyl)phenyl]propanal (origin: Givaudan SA, Vernier, Switzerland), 2,2,2-trichloro-1-phenylethyl acetate.

Preferably, the perfume comprises at least 30%, preferably at least 50%, more preferably at least 60% of ingredients selected from Groups 1 to 7, as defined above. More preferably said perfume comprises at least 30%, preferably at least 50% of ingredients from Groups 3 to 7, as defined above. Most preferably said perfume comprises at least 30%, preferably at least 50% of ingredients from Groups 3, 4, 6 or 7, as defined above.

According to another preferred embodiment, the perfume comprises at least 30%, preferably at least 50%, more preferably at least 60% of ingredients having a log P above 3, preferably above 3.5 and even more preferably above 3.75.

Preferably, the perfume used in the invention contains less than 10% of its own weight of primary alcohols, less than 15% of its own weight of secondary alcohols and less than 20% of its own weight of tertiary alcohols. Advantageously, the perfume used in the invention does not contain any primary alcohols and contains less than 15% of secondary and tertiary alcohols.

According to an embodiment, the oil phase (or the oil-based core) comprises:
25-100 wt % of a perfume oil comprising at least 15 wt % of high impact perfume raw materials having a Log T<−4, and
0-75 wt % of a density balancing material having a density greater than 1.07 g/cm3.

“High impact perfume raw materials” should be understood as perfume raw materials having a Log T<−4. The odor threshold concentration of a chemical compound is determined in part by its shape, polarity, partial charges and molecular mass. For convenience, the threshold concentration is presented as the common logarithm of the threshold concentration, i.e., Log [Threshold] (“Log T”).

A “density balancing material” should be understood as a material having a density preferably greater than 1.07 g/cm3 and having preferably low or no odor.

The density of a component is defined as the ratio between its mass and its volume (g/cm3).

Several methods are available to determine the density of a component.

One may refer for example to the ISO 298:1998 method to measure d20 densities of essential oils.

The odor threshold concentration of a perfuming compound is determined by using a gas chromatograph (“GC”). Specifically, the gas chromatograph is calibrated to determine the exact volume of the perfume oil ingredient injected by the syringe, the precise split ratio, and the hydrocarbon response using a hydrocarbon standard of known concentration and chain-length distribution. The air flow rate is accurately measured and, assuming the duration of a human inhalation to last 12 seconds, the sampled volume is calculated. Since the precise concentration at the detector at any point in time is known, the mass per volume inhaled is known and hence the concentration of the perfuming compound. To determine the threshold concentration, solutions are delivered to the sniff port at the back-calculated concentration. A panelist sniffs the GC effluent and identifies the retention time when odor is noticed. The average across all panelists determines the odor threshold concentration of the perfuming compound. The determination of odor threshold is described in more detail in C. Vuilleumier et al., Multidimensional Visualization of Physical and Perceptual Data Leading to a Creative Approach in Fragrance Development, Perfume & Flavorist, Vol. 33, September, 2008, pages 54-61.

According to an embodiment, the high impact perfume raw materials having a Log T<−4 are selected from the group consisting of (+−)-1-methoxy-3-hexanethiol, 4-(4-hydroxy-1-phenyl)-2-butanone, 2-methoxy-4-(1-propenyl)-1-phenyl acetate, pyrazobutyle, 3-propylphenol, 1-(3-methyl-1-benzofuran-2-yl)ethanone, 2-(3-phenylpropyl)pyridine, 1-(3,3/5,5-dimethyl-1-cyclohexen-1-yl)-4-penten-1-one, 1-(5,5-dimethyl-1-cyclohexen-1-yl)-4-penten-1-one, a mixture comprising (3RS,3aRS,6SR,7ASR)-perhydro-3,6-dimethyl-benzo[b]furan-2-one and (3SR,3aRS,6SR,7ASR)-perhydro-3,6-dimethyl-benzo[b]furan-2-one, (+−)-1-(5-ethyl-5-methyl-1-cyclohexen-1-yl)-4-penten-1-one, (1'S,3′R)-1-methyl-2-[(1′,2′,2′-trimethylbicyclo[3.1.0]hex-3′-yl)methyl]cyclopropyl}methanol, (+−)-3-mercaptohexyl acetate, (2E)-1-(2,6,6-trimethyl-1,3-cyclohexadien-1-yl)-2-buten-1-one, H-methyl-2h-1,5-benzodioxepin-3(4H)-one, (2E,6Z)-2,6-nonadien-1-ol, (4Z)-4-dodecenal, (+−)-4-hydroxy-2,5-dimethyl-3(2H)-furanone, methyl 2,4-dihydroxy-3,6-dimethylbenzoate, 3-methylindole, (+−)-perhydro-4alpha,8abeta-dimethyl-4a-naphthalenol, patchoulol, 2-methoxy-4-(1-propenyl)phenol, mixture comprising (+−)-5,6-dihydro-4-methyl-2-phenyl-2H-pyran and tetrahydro-4-methylene-2-phenyl-2H-pyran, mixture comprising 4-methylene-2-phenyltetrahydro-2H-pyran and (+−)-4-methyl-2-phenyl-3,6-dihydro-2H-pyran, 4-hydroxy-3-methoxybenzaldehyde, nonylenic aldehyde, 2-methoxy-4-propylphenol, 3-methyl-5-phenyl-2-pentenenitrile, 1-(spiro[4.5]dec-6/7-en-7-yl)-4-penten-1-one, 2-methoxynaphthalene, (−)-(3aR,5AS,9AS,9BR)-3a,6,6,9a-tetramethyldodecahydronaphtho[2,1-b]furan, 5-nonanolide, (3aR,5AS,9AS,9BR)-3a,6,6,9a-tetramethyldodecahydronaphtho[2,1-b]furan, 7-isopropyl-2H,4H-1,5-benzodioxepin-3-one, coumarin, 4-methylphenyl isobutyrate, (2E)-1-(2,6,6-trimethyl-1,3-cyclohexadien-1-yl)-2-buten-1-one, beta,2,2,3-tetramethyl-delta-methylene-3-cyclopentene-1-butanol, delta damascone ((2E)-1-[(1RS,2SR)-2,6,6-trimethyl-3-cyclohexen-1-yl]-2-buten-1-one), (+−)-3,6-dihydro-4,6-dimethyl-2-phenyl-2h-pyran, anisaldehyde, paracresol, 3-ethoxy-4-hydroxybenzaldehyde, methyl 2-aminobenzoate, ethyl methylphenylglycidate, octalactone gamma, ethyl 3-phenyl-2-propenoate, (−)-(2E)-2-ethyl-4-[(1R)-2,2,3-trimethyl-3-cyclopenten-1-yl]-2-buten-1-ol, paracresyl acetate, dodecalactone, tricyclone, (+)-(3R,5Z)-3-methyl-5-cyclopentadecen-1-one, undecalactone, (1R,4R)-8-mercapto-3-p-menthanone, (3S,3AS,6R,7AR)-3,6-dimethylhexahydro-1-benzofuran-2(3H)-one, beta ionone, (+−)-6-pentyltetrahydro-2H-pyran-2-one, (3E,5Z)-1,3,5-undecatriene, 10-undecenal, (9E)-9-undecenal (9Z)-9-undecenal, (Z)-4-decenal, (+−)-ethyl 2-methylpentanoate, 1,2-diallyldisulfane, 2-tridecenenitrile, 3-tridecenenitrile, (+−)-2-ethyl-4,4-dimethyl-1,3-oxathiane, (+)-(3R,5Z)-3-methyl-5-cyclopentadecen-1-one, 3-(4-tert-butylphenyl)propanal, allyl (cyclohexyloxy)acetate, methylnaphthylketone, (+−)-(4E)-3-methyl-4-cyclopentadecen-1-one, (+−)-5E3-methyl-5-cyclopentadecen-1-one, cyclopropylmethyl 3-hexenoate, (4E)-4-methyl-5-(4-methylphenyl)-4-pentenal, (+−)-1-(5-propyl-1,3-benzodioxol-2-yl)ethanone, 4-methyl-2-pentylpyridine, (+−)-(E)-3-methyl-4-(2,6,6-trimethyl-2-cyclohexen-1-yl)-3-buten-2-one, (3aRS,5aSR,9aSR,9bRS)-3a,6,6,9a-tetramethyldodecahydronaphtho[2,1-b]furan, (2S,5R)-5-methyl-2-(2-propanyl)cyclohexanone oxime, 6-hexyltetrahydro-2H-pyran-2-one, (+−)-3-(3-isopropyl-1-phenyl)butanal, methyl 2-(3-oxo-2-pentylcyclopentyl)acetate, 1-(2,6,6-trimethyl-1-cyclohex-2-enyl)pent-1-en-3-one, indol, 7-propyl-2H,4H-1,5-benzodioxepin-3-one, ethyl praline, (4-methylphenoxy)acetaldehyde, ethyl tricyclo[5.2.1.0.2,6]decane-2-carboxylate, (+)-(1'S,2S,E)-3,3-dimethyl-5-(2′,2′,3′-trimethyl-3′-cyclopenten-1′-yl)-4-penten-2-ol, (4E)-3,3-dimethyl-5-[(1R)-2,2,3-trimethyl-3-cyclopenten-1-yl]-4-penten-2-ol, 8-isopropyl-6-methyl-bicyclo[2.2.2]oct-5-ene-2-carbaldehyde, methylnonylacetaldehyde, 4-formyl-2-methoxyphenyl 2-methylpropanoate, (E)-4-decenal, (+−)-2-ethyl-4-(2,2,3-trimethyl-3-cyclopenten-1-yl)-2-buten-1-ol, (1R,5R)-4,7,7-trimethyl-6-thiabicyclo[3.2.1]oct-3-ene, (1R,4R,5R)-4,7,7-trimethyl-6-thiabicyclo[3.2.1]octane, (−)-(3R)-3,7-dimethyl-1,6-octadien-3-ol, (E)-3-phenyl-2-propenenitrile, 4-methoxybenzyl acetate, (E)-3-methyl-5-(2,2,3-trimethyl-3-cyclopenten-1-yl)-4-penten-2-ol, allyl (2/3-methylbutoxy)acetate, (+−)-(2E)-1-(2,6,6-trimethyl-2-cyclohexen-1-yl)-2-buten-1-one, (1E)-1-(2,6,6-trimethyl-1-cyclohexen-1-yl)-1-penten-3-one, and mixtures thereof.

According to an embodiment, perfume raw materials having a Log T<−4 are chosen in the group consisting of aldehydes, ketones, alcohols, phenols, esters lactones, ethers, epoxydes, nitriles and mixtures thereof.

According to an embodiment, perfume raw materials having a Log T<−4 comprise at least one compound chosen in the group consisting of alcohols, phenols, esters lactones, ethers, epoxydes, nitriles and mixtures thereof, preferably in amount comprised between 20 and 70 wt. % based on the total weight of the perfume raw materials having a Log T<−4.

According to an embodiment, perfume raw materials having a Log T<−4 comprise between 20 and 70 wt. % by weight of aldehydes, ketones, and mixtures thereof based on the total weight of the perfume raw materials having a Log T<−4.

The remaining perfume raw materials contained in the oil-based core may have therefore a Log T>−4.

According to an embodiment, the perfume raw materials having a Log T>−4 are chosen in the group consisting of ethyl 2-methylbutyrate, (E)-3-phenyl-2-propenyl acetate, (+−)-6/8-sec-butylquinoline, (+−)-3-(1,3-benzodioxol-5-yl)-2-methylpropanal, verdyl propionate, 1-(octahydro-2,3,8,8-tetramethyl-2-naphtalenyl)-1-ethanone, methyl 2-((1RS,2RS)-3-oxo-2-pentylcyclopentyl)acetate, (+−)-(E)-4-methyl-3-decen-5-ol, 2,4-dimethyl-3-cyclohexene-1-carbaldehyde, 1,3,3-trimethyl-2-oxabicyclo[2.2.2]octane, tetrahydro-4-methyl-2-(2-methyl-1-propenyl)-2H-pyran, dodecanal, 1-oxa-12/13-cyclohexadecen-2-one, (+−)-3-(4-isopropylphenyl)-2-methylpropanal, aldehyde C11, (+−)-2,6-dimethyl-7-octen-2-ol, allyl 3-cyclohexylpropanoate, (Z)-3-hexenyl acetate, 5-methyl-2-(2-propanyl)cyclohexanone, allyl heptanoate, 2-(2-methyl-2-propanyl)cyclohexyl acetate, 1,1-dimethyl-2-phenylethyl butyrate, geranyl acetate, neryl acetate, (+−)-1-phenylethyl acetate, 1,1-dimethyl-2-phenylethyl acetate, 3-methyl-2-butenyl acetate, ethyl 3-oxobutanoate, (2Z)-ethyl 3-hydroxy-2-butenoate, 8-p-menthanol, 8-p-menthanyl acetate, 1-p-menthanyl acetate, (+−)-2-(4-methyl-3-cyclohexen-1-yl)-2-propanyl acetate, (+−)-2-methylbutyl butanoate, 2-{(1S)-1-[(1R)-3,3-dimethylcyclohexyl]ethoxy}-2-oxoethyl propionate, 3,5,6-trimethyl-3-cyclohexene-1-carbaldehyde, 2,4,6-trimethyl-3-cyclohexene-1-carbaldehyde, 2-cyclohexylethyl acetate, octanal, ethyl butanoate, (+−)-(3E)-4-(2,6,6-trimethyl-1/2-cyclohexen-1-yl)-3-buten-2-one, 1-[(1RS,6SR)-2,2,6-trimethylcyclohexyl]-3-hexanol, 1,3,3-trimethyl-2-oxabicyclo[2.2.2]octane, 1,3,3-trimethyl-2-oxabicyclo[2.2.2]octane, ethyl hexanoate, undecanal, decanal, 2-phenylethyl acetate, (1S,2S,4S)-1,7,7-trimethylbicyclo[2.2.1]heptan-2-ol, (1S,2R,4S)-1,7,7-trimethylbicyclo[2.2.1]heptan-2-ol), (+−)-3,7-dimethyl-3-octanol, 1-methyl-4-(2-propanylidene)cyclohexene, (+)-(R)-4-(2-methoxypropan-2-yl)-1-methylcyclohex-1-ene, verdyl acetate, (3R)-1-[(1R,6S)-2,2,6-trimethylcyclohexyl]-3-hexanol, (3S)-1-[(1R,6S)-2,2,6-trimethylcyclohexyl]-3-hexanol, (3R)-1-[(1S,6S)-2,2,6-trimethylcyclohexyl]-3-hexanol, (+)-(1S,1′R)-2-[1-(3′,3′-dimethyl-1-cyclohexyl)ethoxy]-2-methylpropyl propanoate, and mixtures thereof.

The nature of high impact perfume raw materials having a Log T<−4 and density balancing material having a density greater than 1.07 g/cm3 are described in WO2018115250, the content of which are included by reference.

The term “biocide” refers to a chemical substance capable of killing living organisms (e.g. microorganisms) or reducing or preventing their growth and/or accumulation. Biocides are commonly used in medicine, agriculture, forestry, and in industry where they prevent the fouling of, for example, water, agricultural products including seed, and oil pipelines. A biocide can be a pesticide, including a fungicide, herbicide, insecticide, algicide, molluscicide, miticide and rodenticide; and/or an antimicrobial such as a germicide, antibiotic, antibacterial, antiviral, antifungal, antiprotozoal and/or antiparasite.

As used herein, a “pest control agent” indicates a substance that serves to repel or attract pests, to decrease, inhibit or promote their growth, development or their activity. Pests refer to any living organism, whether animal, plant or fungus, which is invasive or troublesome to plants or animals, pests include insects notably arthropods, mites, spiders, fungi, weeds, bacteria and other microorganisms.

According to an embodiment, the perfume formulation comprises
0 to 60 wt. % of a hydrophobic solvent (based on the total weight of the perfume formulation),
40 to 100 wt. % of a perfume oil (based on the total weight of the perfume formulation), wherein the perfume oil has at least two, preferably all of the following characteristics:
at least 35%, preferably at least 40%, preferably at least 50%, more preferably at least 60% of perfuming ingredients having a log P above 3, preferably above 3.5,
at least 20%, preferably at least 25%, preferably at least 30%, more preferably at least 40% of Bulky materials of groups 1 to 6, preferably 3 to 6 as previously defined and
at least 15%, preferably at least 20%, more preferably at least 25%, even more preferably at least 30% of high impact perfume materials having a Log T<−4 as previously defined,
optionally, further hydrophobic active ingredients.

According to a particular embodiment, the perfume comprises 0 to 60 wt. % of a hydrophobic solvent.

According to a particular embodiment, the hydrophobic solvent is a density balancing material preferably chosen in the group consisting of benzyl salicylate, benzyl benzoate, cyclohexyl salicylate, benzyl phenylacetate, phenylethyl phenylacetate, triacetin, ethyl citrate, methyl and ethyl salicylate, benzyl cinnamate, and mixtures thereof.

In a particular embodiment, the hydrophobic solvent has Hansen Solubility Parameters compatible with entrapped perfume oil.

The term “Hansen solubility parameter” is understood refers to a solubility parameter approach proposed by Charles Hansen used to predict polymer solubility and was developed around the basis that the total energy of vaporization of a liquid consists of several individual parts. To calculate the “weighted Hansen solubility parameter” one must combine the effects of (atomic) dispersion forces, (molecular) permanent dipole-permanent dipole forces, and (molecular) hydrogen bonding (electron exchange). The weighted Hansen solubility parameter” is calculated as (δD2+δP2+δH2)0.5, wherein δD is the Hansen dispersion value (also referred to in the following as the atomic dispersion fore), bP is the Hansen polarizability value (also referred to in the following as the dipole moment), and bH is the Hansen Hydrogen-bonding (“h-bonding”) value (also referred to in the following as hydrogen bonding). For a more detailed description of the parameters and values, see Charles Hansen, The Three Dimensional Solubility Parameter and Solvent Diffusion Coefficient, Danish Technical Press (Copenhagen, 1967).

Euclidean difference in solubility parameter between a fragrance and a solvent is calculated as (4*(δDsolvent−δDfragrance)2+(δPsolvent−δPfragrance)2+(δHsolvent−δHfragrance)2)0.5, in which δDsolvent, δPsolvent, and δHsolvent, are the Hansen dispersion value, Hansen polarizability value, and Hansen h-bonding values of the solvent, respectively; and δDfragrance, δPfragrance, and δHfragrance are the Hansen dispersion value, Hansen polarizability value, and Hansen h-bonding values of the fragrance, respectively.

In a particular embodiment, the perfume oil and the hydrophobic solvent have at least two Hansen solubility parameters selected from a first group consisting of: an atomic dispersion force (δD) from 12 to 20, a dipole moment (δP) from 1 to 8, and a hydrogen bonding (δH) from 2.5 to 11.

In a particular embodiment, the perfume oil and the hydrophobic solvent have at least two Hansen solubility parameters selected from a second group consisting of: an atomic dispersion force (δD) from 12 to 20, preferably from 14 to 20, a dipole moment (δP) from 1 to 8, preferably from 1 to 7, and a hydrogen bonding (δH) from 2.5 to 11, preferably from 4 to 11 According to a particular embodiment, the hydrophobic material is free of any active ingredient (such as perfume). According to this particular embodiment, it comprises, preferably consists of hydrophobic solvents, preferably chosen in the group consisting of isopropyl myristate, tryglycerides (e.g. Neobee® MCT oil, vegetable oils), D-limonene, silicone oil, mineral oil, and mixtures thereof with optionally hydrophilic solvents preferably chosen in the group consisting of 1,4-butanediol, benzyl alcohol, triethyl citrate, triacetin, benzyl acetate, ethyl acetate, propylene glycol (1,2-propanediol), 1,3-propanediol, dipropylene glycol, glycerol, glycol ethers and mixtures thereof.

According to any one of the invention's embodiments, the hydrophobic material represents between about 10% and 90% w/w, preferably between 10 and 60%, or even between 15% and 45% w/w, by weight, relative to the total weight of the oil phase.

### Polymeric Shell

According to the present invention, the core-shell microcapsule comprises a polymeric shell.

By the expression polymeric shell is understood that the shell comprises at least one polymer forming a surrounding structure of the core.

The nature of the polymeric shell of the microcapsules of the invention can vary.

As non-limiting examples, the polymer shell comprises a material selected from the group consisting of polyurea, polyurethane, polyamide, polyhydroxyalkanoates, polyacrylate, polyesters, polyaminoesters, polyepoxides, polysiloxane, polycarbonate, polysulfonamide, urea formaldehyde, melamine formaldehyde resin, melamine formaldehyde resin cross-linked with polyisocyanate or aromatic polyols, melamine urea resin, melamine glyoxal resin, gelatin/gum arabic, and mixtures thereof.

The material encapsulating the hydrophobic material composition can be microcapsules which have been widely described in the prior art.

In a first particular embodiment of the core-shell microcapsules, the core-shell microcapsule comprises an oil-based core comprising a hydrophobic active, preferably perfume, and a composite shell comprising a first material and a second material, wherein the first material and the second material are different, the first material is a coacervate, the second material is a polymeric material.

In a particular embodiment, the weight ratio between the first material and the second material is comprised between 50:50 and 99.9:0.1.

In a particular embodiment, the coacervate comprises a first polyelectrolyte, preferably selected among proteins (such as gelatin), polypeptides or polysaccharides (such as chitosan), most preferably Gelatin and a second polyelectrolyte, preferably alginate salts, cellulose derivatives guar gum, pectinate salts, carrageenan, polyacrylic and methacrylic acid or xanthan gum, or yet plant gums such as acacia gum (Gum Arabic), most preferably Gum Arabic.

The coacervate first material can be hardened chemically using a suitable cross-linker such as glutaraldehyde, glyoxal, formaldehyde, tannic acid or genipin or can be hardened enzymatically using an enzyme such as transglutaminase.

The second polymeric material can be selected from the group consisting of polyurea, polyurethane, polyamide, polyester, polyacrylate, polysiloxane, polycarbonate, polysulfonamide, polymers of urea and formaldehyde, melamine and formaldehyde, melamine and urea, or melamine and glyoxal and mixtures thereof, preferably polyurea and/or polyurethane. The second material is preferably present in an amount less than 3 wt. %, preferably less than 1 wt. % based on the total weight of the microcapsule slurry.

As non-limiting examples, the shell can be aminoplast-based, polyurea-based or polyurethane-based. The shell can also be hybrid, namely organic-inorganic such as a hybrid shell composed of at least two types of inorganic particles that are cross-linked, or yet a shell resulting from the hydrolysis and condensation reaction of a polyalkoxysilane macro-monomeric composition.

According to an aspect, the shell comprises an aminoplast copolymer, such as melamine-formaldehyde or urea-formaldehyde or cross-linked melamine formaldehyde or melamine glyoxal.

According to another aspect the shell is polyurea-based made from, for example but not limited to isocyanate-based monomers and amine-containing crosslinkers such as guanidine carbonate and/or guanazole. Certain polyurea microcapsules comprise a polyurea wall which is the reaction product of the polymerisation between at least one polyisocyanate comprising at least two isocyanate functional groups and at least one reactant selected from the group consisting of an amine (for example a water-soluble guanidine salt and guanidine); a colloidal stabilizer or emulsifier; and an encapsulated perfume. However, the use of an amine can be omitted.

According to a particular aspect, the colloidal stabilizer includes an aqueous solution of between 0.1% and 0.4% of polyvinyl alcohol, between 0.6% and 1% of a cationic copolymer of vinylpyrrolidone and of a quaternized vinylimidazol (all percentages being defined by weight relative to the total weight of the colloidal stabilizer). According to another aspect, the emulsifier is an anionic or amphiphilic biopolymer, which may be, in one aspect, chosen from the group consisting of gum Arabic, soy protein, gelatin, sodium caseinate and mixtures thereof.

According to another aspect, the shell is polyurethane-based made from, for example but not limited to polyisocyanate and polyols, polyamide, polyester, etc.

In one aspect, the microcapsule wall material may comprise any suitable resin and especially including melamine, glyoxal, polyurea, polyurethane, polyamide, polyester, etc. Suitable resins include the reaction product of an aldehyde and an amine, suitable aldehydes include, formaldehyde and glyoxal. Suitable amines include melamine, urea, benzoguanamine, glycoluril, and mixtures thereof. Suitable melamines include, methylol melamine, methylated methylol melamine, imino melamine and mixtures thereof. Suitable ureas include, dimethylol urea, methylated dimethylol urea, urea-resorcinol, and mixtures thereof. Suitable materials for making may be obtained from one or more of the following companies Solutia Inc. (St Louis, Missouri U.S.A.), Cytec Industries (West Paterson, New Jersey U.S.A.), Sigma-Aldrich (St. Louis, Missouri U.S.A.).

According to one aspect, the microcapsule is a one-shell aminoplast core-shell microcapsule obtainable by a process comprising the steps of:
1) admixing a perfume oil with at least a polyisocyanate having at least two isocyanate functional groups to form an oil phase;
2) dispersing or dissolving into water an aminoplast resin and optionally a stabilizer to form a water phase;
3) preparing an oil-in-water dispersion, wherein the mean droplet size is comprised between 1 and 100 microns, by admixing the oil phase and the water phase;
4) performing a curing step to form the wall of said microcapsule; and
5) optionally drying the final dispersion to obtain the dried core-shell microcapsule.

According to one aspect, the core-shell microcapsule is a formaldehyde-free capsule. A typical process for the preparation of aminoplast formaldehyde-free microcapsules slurry comprises the steps of
1) preparing an oligomeric composition comprising the reaction product of, or obtainable by reacting together:
   a. a polyamine component in the form of melamine or of a mixture of melamine and at least one C₁-C₄ compound comprising two NH2 functional groups;
   b. an aldehyde component in the form of a mixture of glyoxal, a C₄₋₆ 2,2-dialkoxy-ethanal and optionally a glyoxalate, said mixture having a molar ratio glyoxal/C₄₋₆ 2,2-dialkoxy-ethanal comprised between 1/1 and 10/1; and
   c. a protic acid catalyst;
2) preparing an oil-in-water dispersion, wherein the droplet size is comprised between 1 and 600 microns, and comprising:
   a. an oil;
   b. a water medium:
   c. at least an oligomeric composition as obtained in step 1;
   d. at least a cross-linker selected amongst:
      i. C₄-C₁₂ aromatic or aliphatic di- or tri-isocyanates and their biurets, triurets, trimmers, trimethylol propane-adduct and mixtures thereof; and/or
      ii. a di- or tri-oxiran compounds of formula:

A-(oxiran-2-ylmethyl)ₙ
whereinₙ stands for 2 or 3 and 1 represents a C₂-C₆ group optionally comprising from 2 to 6 nitrogen and/or oxygen atoms;
e. optionally a C₁-C₄ compounds comprising two NH₂ functional groups;
3) Heating the dispersion; and
4) Cooling the dispersion.

The above process is described in more details in International Patent Application Publication No. WO 2013/068255.

In an another particular embodiment of the core-shell microcapsules, the core-shell microcapsule comprises
an oil-based core comprising a hydrophobic active, preferably perfume,
optionally an inner shell made of a polymerized polyfunctional monomer;
a biopolymer shell comprising a protein, wherein at least one protein is cross-linked.

According to a particular embodiment, the protein is chosen in the group consisting of milk proteins, caseinate salts such as sodium caseinate or calcium caseinate, casein, whey protein, hydrolyzed proteins, gelatins, gluten, pea protein, soy protein, silk protein and mixtures thereof, preferably sodium caseinate, most preferably sodium caseinate

According to a particular embodiment, the protein comprises sodium caseinate and a globular protein, preferably chosen in the group consisting of whey protein, beta-lactoglobulin, ovalbumine, bovine serum albumin, vegetable proteins, and mixtures thereof.

The protein is preferably a mixture of sodium caseinate and whey protein.

According to a particular embodiment, the biopolymer shell comprises a crosslinked protein chosen in the group consisting of sodium caseinate and/or whey protein.

According to a particular embodiment, the microcapsules slurry comprises at least one microcapsule made of:
an oil-based core comprising the hydrophobic active, preferably perfume;
an inner shell made of a polymerized polyfunctional monomer; preferably a polyisocyanate having at least two isocyanate functional groups
a biopolymer shell comprising a protein, wherein at least one protein is cross-linked;
wherein the protein contains preferably a mixture comprising sodium caseinate and a globular protein, preferably whey protein.
optionally at least an outer mineral layer.

According to an embodiment, sodium caseinate and/or whey protein is (are) cross-linked protein(s).

The weight ratio between sodium caseinate and whey protein is preferably comprised between 0.01 and 100, preferably between 0.1 and 10, more preferably between 0.2 and 5.

In another particular embodiment of the core-shell microcapsules, the core-shell microcapsule is a polyamide core-shell polyamide microcapsule comprising:
an oil-based core comprising comprising a hydrophobic active, preferably perfume, and
a polyamide shell comprising or being obtainable from:
   an acyl chloride,
   a first amino compound, and
   a second amino compound.

According to a particular embodiment, the polyamide core-shell microcapsule comprises:
an oil-based core comprising a hydrophobic active, preferably perfume, and
a polyamide shell comprising or being obtainable from:
   an acyl chloride, preferably in an amount comprised between 5 and 98%, preferably between 20 and 98%, more preferably between 30 and 85% w/w
   a first amino compound, preferably in an amount comprised between 1% and 50% w/w, preferably between 7 and 40% w/w;
   a second amino compound, preferably in an amount comprised between 1% and 50% w/w, preferably between 2 and 25% w/w
   a stabilizer, preferably a biopolymer, preferably in an amount comprised between 0 and 90%, preferably between 0.1 and 75%, more preferably between 1 and 70%.

According to a particular embodiment, the polyamide core-shell microcapsule comprises:
an oil-based core comprising a hydrophobic active, preferably perfume, and
a polyamide shell comprising or being obtainable from:
   an acyl chloride,
   a first amino-compound being an amino-acid, preferably chosen in the group consisting of L-Lysine, L-Arginine, L-Histidine, L-Tryptophane and/or mixture thereof.
   a second amino compound chosen in the group consisting of ethylene diamine, diethylene triamine, cystamine and/or mixture thereof, and
   a biopolymer chosen in the group consisting of casein, sodium caseinate, bovin serum albumin, whey protein, and/or mixture thereof.

The first amino-compound can be different from the second amino-compound.

According to another aspect, the shell of the microcapsule is polyurea- or polyurethane-based. Examples of processes for the preparation of polyurea and polyurethane-based microcapsule slurry are for instance described in International Patent Application Publication No. WO2007/004166, European Patent Application Publication No. EP 2300146, and European Patent Application Publication No. EP25799. Typically a process for the preparation of polyurea or polyurethane-based microcapsule slurry include the following steps:
a) Dissolving at least one polyisocyanate having at least two isocyanate groups in an oil to form an oil phase;
b) Preparing an aqueous solution of an emulsifier or colloidal stabilizer to form a water phase;
c) Adding the oil phase to the water phase to form an oil-in-water dispersion, wherein the mean droplet size is comprised between 1 and 500 μm, preferably between 5 and 50 μm; and
d) Applying conditions sufficient to induce interfacial polymerisation and form microcapsules in form of a slurry.

In a particular embodiment, the shell material is a biodegradable material.

In a particular embodiment, the shell has a biodegradability of at least 40%, preferably at least 60%, 65%, 70%, 75%, 80%, 85%, 90%, 95% or 98%, within 60 days according to OECD301F.

In a particular embodiment, the core-shell microcapsule has a biodegradability of at least 40%, preferably at least 60%, preferably at least 65%, 70%, 75%, 80%, 85%, 90%, 95% or 98% within 60 days according to OECD301F.

Thereby it is understood that the core-shell microcapsule including all components, such as the core, shell and coating may have a biodegradability of at least 40%, preferably at least 60%, preferably at least 65%, 70%, 75%, 80%, 85%, 90%, 95% or 98% within 60 days according to OECD301F.

In a particular embodiment, the oil-based core, preferably perfume oil has a biodegradability of at least 40%, preferably at least 60%, preferably at least 65%, 70%, 75%, 80%, 85%, 90%, 95% or 98% within 60 days according to OECD301F.

OECD301F is a standard test method on the biodegradability from the Organization of Economic Co-operation and Development.

A typical method for extracting the shell for measuring the biodegradability is disclosed in Gasparini and all in Molecules 2020, 25,718.

### First Deposition Aid

According to the present invention, the microcapsule comprises a coating comprising a first deposition aid.

Under a “deposition aid”, an aid is understood that increases the capability of the microcapsules to deposit on a target substrate such as textile, skin, hair or other surfaces. Hence, a deposition aid increases the capability of the microcapsules to deposit on the intended target site where the microcapsules are supposed to exert their effect.

The expression coating is herein understood in that the first deposition aid is surrounding the polymer shell by means of non-chemical interaction, such as physical adsorption and/or electrostatic interaction, or chemical bonding between the coating and the polymeric shell, such as grafting, preferably by means of non-chemical interaction.

In a particular embodiment, the coating forms a second shell-like structure around the polymeric shell.

In a particular embodiment, the first and second deposition aid have opposite net charges at a pH when solubilized. In a particular embodiment, the first deposition aid has a positive net charge at a pH of less than 8 and the second deposition aid have a negative net charge at a pH of more than 2. The net charge is measured by measuring the Zeta potential. The zeta potential can be measure for example by the Malvern Zetasizer.

In a particular embodiment, the first deposition aid has a positive net charge at neutral or acidic pH. In a particular embodiment, the first deposition aid has a positive net charge at a pH of less than 8.

In a particular embodiment, the first deposition aid is positively charged for pH<8 so as to form gels or highly viscous solutions in water below the gelling temperature, and lower viscosity solutions in water at a temperature above the melting point of the gel. The viscosity above the gelling temperature is typically lower than 0.1 Pa s; below the gelling temperature, the elastic modulus G′ of the gel is typically in the range 0.1-15 kPa when measured during the first 24 hours after gel formation, using the measurement methods based on shear rheometry (such methods, along with the definitions relevant for the gelling temperature, are described, for example, in Parker, A. and Normand, V., Soft Matter, 6, pp 4916-4919 (2010).

In a particular embodiment, the first deposition aid comprises a biopolymer or is biopolymer based.

By biopolymers it is herein understood biomacromolecules produced by living organisms. Biopolymers are characterized by molecular weight distributions ranging from 1,000 (1 thousand) to 1,000,000,000 (1 billion) Daltons. These macromolecules may be carbohydrates (sugar based) or proteins (amino-acid based) or a combination of both (gums) and can be linear or branched.

In a particular embodiment, the biopolymers have not been modified by means of chemical derivatization to chemically graft on different functional groups with different properties.

In a particular embodiment, the biopolymer-based deposition aids are based on a biopolymer and have been further modified by means of chemical derivatization to chemically graft on different functional groups with different properties.

In a particular embodiment, the first deposition aid comprises chitosan or a functionalized chitosan derivative.

The term chitosan in turn is understood as a linear polysaccharide composed of D-glucosamine monomers (deacetylated unit) and, optionally, randomly distributed N-acetyl-D-glucosamine monomers (acetylated unit).

The expression chitosan derivative is herein understood as being based on chitosan.

In a particular embodiment, the chitosan or chitosan of the chitosan derivative has a degree of deacetylation (DD %) of 50% or more. In a particular embodiment, the chitosan or chitosan of the chitosan derivative has a degree of deacetylation of 60% or more, preferably of 70% or more or more preferably of 80% or more. The degree of deacetylation can be determined by NMR spectroscopy, in particular solid state ¹³C NMR spectroscopy.

In a particular embodiment, the chitosan or functionalized chitosan derivative has a molecular weight Mw from 3 kDa to 5 MDa, preferably from 900 kDa to 4 MDa, even more preferably 1 MDa to 3.5 MDa, even more preferably 1.25 MDa to 1.8 MDa.

By the expression functionalized chitosan derivative, it is herein understood that chitosan is modified by a functional group, e.g. functionalized by a cationic agent, a hydrophobic agent, a catechol group containing agent, an anionic agent and/or thiolating agent linked to the chitosan backbone. In particular, it is understood that chitosan is modified by a functional group which is not already present in natural chitosan. In particular it is understood that a functionalization is not a deacetylation of potentially remaining acetyl groups from chitosan and/or a rearrangement/hydrolysis of the chitosan backbone. In particular it is also understood that the functionalization of the chitosan does not comprise a functionalization with acetyl groups, i.e. the functionalized chitosan derivative is not chitin. In particular it is also understood that the functionalization of the chitosan does not relate to protonation of the amino function of gluocosamine natural chitosan.

In a particular embodiment, the functionalized chitosan derivative is obtained by chemical or biotechnological functionalization of chitosan, preferably obtained by chemical functionalization.

In a particular embodiment, the functionalized chitosan derivative is obtained by chemical or biotechnological functionalization of chitosan, preferably obtained by chemical functionalization, e.g. by functionalization with a cationic agent, a hydrophobic agent, a catechol group containing agent, an anionic agent and/or thiolating agent chemically or biotechnologically linked to the chitosan backbone.

In a particular embodiment, the functionalized chitosan derivative is not obtained by protonation of the amino function of gluocosamine of natural chitosan.

In a particular embodiment, the functionalized chitosan derivative has a degree of deacetylation (DD %) of 50% or more. In a particular embodiment, the functionalized chitosan derivative has a degree of deacetylation of 60% or more, preferably 70% or more, more preferably 80% or more.

In a particular embodiment, the functionalized chitosan derivative is functionalized to a degree from 10% to 100% of the amino groups. In a particular embodiment, the functionalized chitosan derivative may be functionalized to a degree of at least 40%, preferably 60%, more preferably at least 80%. In a particular embodiment, the functionalized chitosan may be functionalized to maximum 100% or maximum 99%. The degree of functionalization can be determined by NMR, in particular solid state ¹³C NMR spectroscopy.

In a particular embodiment, the functionalized chitosan derivative has a molecular weight M_{W} of at least 5 kDa, preferably at least 1 MDa. In a particular embodiment, the functionalized chitosan derivative has a molecular weight of from 800 kDa to 5 MDa, preferably from 1 MDa to 4 MDa.

In a particular embodiment, the at least one core-shell microcapsule comprising the coating has a positive zeta potential. In a particular embodiment, the at least one core-shell microcapsule comprising the coating has a zeta potential of +35 to +85 mV. The zeta potential can be measured for example by the Malvern Zetasizer.

In a particular embodiment, the functionalized chitosan derivative is functionalized with a cationic agent, a hydrophobic agent, a catechol group containing agent, an anionic agent and/or thiolating agent linked to the chitosan backbone.

In a particular embodiment, the functionalized chitosan derivative is functionalized with a cationic agent, a hydrophobic agent and/or an anionic agent linked to the chitosan backbone.

In a particular embodiment, the cationic agent, a hydrophobic agent, a catechol group containing agent, an anionic agent and/or thiolating agent is directly linked to the chitosan backbone or linked by means of a linker group, preferably an organic linker group. A person skilled in the art is aware of linker groups.

In a particular embodiment, the functionalized chitosan derivative is functionalized with glycidyl trimethylammonium chloride, 3-chloro-2-hydroxypropyltrimethylammonium chloride, (2-octen-1-yl)succinic anhydride, (2-dodecen-1-yl) succinic anhydride, succinic anhydride, maleic anhydride, 3,4-dihydroxyphenylacetic acid, 3,4-dihydroxyhydrocinnamic acid, 2-mercaptoacetic acid, 3-mercaptopropanoic acid linked to the chitosan backbone.

In a particular embodiment, the first deposition aid, preferably chitosan or functionalized chitosan derivative, is not crosslinked with the polymeric shell.

According to a particular embodiment, the coating of the core-shell microcapsule may comprise an additional coating material selected from the group consisting of a non-ionic polysaccharide, a cationic polymer, a polysuccinimide derivative (as described for instance in WO2021185724) and mixtures thereof to form the outer coating to the microcapsule.

Non-ionic polysaccharide polymers are well known to a person skilled in the art. Preferred non-ionic polysaccharides are selected from the group consisting of locust bean gum, xyloglucan, guar gum, hydroxypropyl guar, hydroxypropyl cellulose and hydroxypropyl methyl cellulose, pectin and mixtures thereof.

Cationic polymers are also well known to a person skilled in the art. Preferred cationic polymers have cationic charge densities of at least 0.5 meq/g, more preferably at least about 1.5 meq/g, but also preferably less than about 7 meq/g, more preferably less than about 6.2 meq/g. The cationic charge density of the cationic polymers may be determined by the Kjeldahl method as described in the US Pharmacopoeia under chemical tests for Nitrogen determination. The preferred cationic polymers are chosen from those that contain units comprising primary, secondary, tertiary and/or quaternary amine groups that can either form part of the main polymer chain or can be borne by a side substituent directly connected thereto. The weight average (Mw) molecular weight of the cationic polymer is preferably between 10,000 and 3.5M Dalton, more preferably between 50,000 and 2M Dalton.

According to a particular embodiment, one will use cationic polymers based on acrylamide, methacrylamide, N-vinylpyrrolidone, quaternized N,N-dimethylaminomethacrylate, diallyldimethylammonium chloride, quaternized vinylimidazole (3-methyl-1-vinyl-1H-imidazol-3-ium chloride), vinylpyrrolidone, acrylamidopropyltrimonium chloride, cassia hydroxypropyltrimonium chloride, guar hydroxypropyltrimonium chloride or polygalactomannan 2-hydroxypropyltrimethylammonium chloride ether, starch hydroxypropyltrimonium chloride and cellulose hydroxypropyltrimonium chloride. Preferably copolymers shall be selected from the group consisting of polyquaternium-5, polyquaternium-6, polyquaternium-7, polyquaternium10, polyquaternium-11, polyquaternium-16, polyquaternium-22, polyquaternium-28, polyquaternium-43, polyquaternium-44, polyquaternium-46, cassia hydroxypropyltrimonium chloride, guar hydroxypropyltrimonium chloride or polygalactomannan 2-hydroxypropyltrimethylammonium chloride ether, starch hydroxypropyltrimonium chloride and cellulose hydroxypropyltrimonium chloride

As specific examples of commercially available products, one may cite Salcare® SC60 (cationic copolymer of acrylamidopropyltrimonium chloride and acrylamide, origin: BASF) or Luviquat®, such as the PQ 11N, FC 550 or Style (polyquaternium-11 to 68 or quaternized copolymers of vinylpyrrolidone origin: BASF), or also the Jaguar® (C13S or C17, origin Rhodia).

According to any one of the above embodiments of the invention, there is added an amount of polymer described above comprised between about 0% and 5% w/w, or even between about 0.1% and 2% w/w, percentage being expressed on a w/w basis relative to the total weight of microcapsule slurry. It is clearly understood by a person skilled in the art that only part of said added polymers will be incorporated into/deposited on the microcapsule shell.

In a particular embodiment, the at least one core-shell microcapsule comprises a first coating comprising the first deposition aid adjacent to the polymeric shell.

### Second Deposition Aid

According to the present invention, the core-shell microcapsule slurry comprises a second deposition aid.

It is understood that the first and second deposition aid are different.

In a particular embodiment, the second deposition aid has a negative net charge at neutral or basic pH. In a particular embodiment, the second deposition aid has a negative net charge at a pH of more than 2.

In a particular embodiment, the second deposition aid comprises a biopolymer.

In a particular embodiment, the second deposition aid comprises proteins, polysaccharides, and mixtures thereof.

In a particular embodiment, the second deposition aid comprises alginate, pectin, carboxymethyl-cellulose, whey protein, gum arabic.

The weight ratio between first and second deposition aid is preferably comprised between 10/0.1 to 0.1/10.

In a particular embodiment, the second deposition aid is present in the coating. In said embodiment, the second deposition aid is thus present in the same coating as the first deposition aid.

In a particular embodiment, the at least one core-shell microcapsule comprises a second coating comprising a second deposition aid not adjacent to the polymeric shell.

In a particular embodiment, the at least one core-shell microcapsule comprises a first coating comprising the first deposition aid adjacent to the polymeric shell and a second coating comprising a second deposition aid adjacent to the first coating.

### Optional Components

When microcapsules are in the form of a slurry, the microcapsule slurry can comprise auxiliary ingredients selected from the group of thickening agents/rheology modifiers, antimicrobial agents, opacity-building agents, mica particles, salt, pH stabilizers/buffering ingredients, preferably in an amount comprised between 0 and 15% by weight based on the total weight of the slurry.

According to another embodiment, the microcapsule slurry of the invention comprises additional free (i.e non-encapsulated) perfume, preferably in an amount comprised between 5 and 50% by weight based on the total weight of the slurry.

In a particular embodiment, the core-shell microcapsules are isolated by drying the obtained core-shell microcapsule slurry. Drying can be achieved by submitting the obtained core-shell microcapsule slurry to a drying step, such as spray-drying, to provide the microcapsules as such, i.e. in a powdery form.

It is understood that any standard method known by a person skilled in the art to perform such drying is also applicable. In particular the slurry may be spray-dried preferably in the presence of a polymeric carrier material such as polyvinyl acetate, polyvinyl alcohol, dextrins, natural or modified starch, vegetable gums, pectins, xanthans, alginates, carragenans or cellulose derivatives to provide microcapsules in a powder form.

### Multiple Capsules System

According to an embodiment, the microcapsules of the invention (first type of microcapsule) can be used in combination with a second type of microcapsules.

Another object of the invention is a microcapsule delivery system comprising:
the microcapsules of the present invention as a first type of microcapsules, and
a second type of microcapsules, wherein the first type of microcapsules and the second type of microcapsules differ in their hydrophobic material and/or and/or carrier material (shell or matrix) and/or in their coating material.

### Perfuming Composition/Consumer Products

The microcapsules of the invention can be used in combination with active ingredients. An object of the invention is therefore a composition comprising:
(i) a core-shell microcapsule slurry as described herein-above or a core-shell microcapsule as described herein-above;
(ii) an active ingredient, preferably chosen in the group consisting of a cosmetic ingredient, skin caring ingredient, perfume ingredient, flavor ingredient, malodour counteracting ingredient, bactericide ingredient, fungicide ingredient, pharmaceutical or agrochemical ingredient, a sanitizing ingredient, an insect repellent or attractant, and mixtures thereof.

The present invention also relates to a perfuming composition comprising
a) a core-shell microcapsule slurry as described herein-above,
b) optionally an active ingredient,
c) at least one ingredient selected from the group consisting of a perfumery carrier and a perfumery base,
d) optionally, at least one perfumery adjuvant.

The embodiments and definitions for the oil-based core comprising a hydrophobic material, the polymeric shell, the first and second deposition aid as described herein-above applies mutatis mutandis to the core-shell microcapsules per se.

The perfuming composition may comprise the core-shell microcapsule slurry or core-shell microcapsule between 0.1 and 30 wt. %, based on the total weight of the perfuming composition.

The perfuming composition may further comprise an active ingredient. The active ingredient may preferably be chosen in the group consisting of a cosmetic ingredient, skin caring ingredient, perfume ingredient, flavor ingredient, malodour counteracting ingredient, bactericide ingredient, fungicide ingredient, pharmaceutical or agrochemical ingredient, a sanitizing ingredient, an insect repellent or attractant, and mixtures thereof.

In a particular embodiment, the perfuming composition comprises a free perfume oil.

By “free perfume” it is herein understood a perfume or perfume oil which is comprised in the perfuming composition and not entrapped in the core-shell microcapsule.

The perfuming composition may comprise the active ingredient, preferably the free perfume, between 0.1 and 30 wt. %, based on the total weight of the perfuming composition.

In a particular embodiment, the total amount of the microcapsule slurry or microcapsule is 0.05 to 5 wt. %, based on the total weight of the perfuming composition, and the total amount of the free perfume oil is 0.05 to 5 wt. %, based on the total weight of the perfuming composition.

In a particular embodiment, the total perfume oil of the perfume formulation entrapped in the core-shell microcapsule and total free perfume oil are present in the perfuming composition in a weight ratio of 1:20 to 20:1, preferably 10:1 to 1:10.

The perfuming composition can further comprise at least one perfuming co-ingredient and, optionally a perfumery adjuvant.

By “perfuming co-ingredient” it is herein understood a compound, which is used in a perfuming preparation or a composition to impart a hedonic effect and which is not a microcapsule as 20 defined above. In other words such a co-ingredient, to be considered as being a perfuming one, must be recognized by a person skilled in the art as being able to impart or modify in a positive or pleasant way the odor of a composition, and not just as having an odor. The nature and type of the perfuming co-ingredients present in the perfuming composition do not warrant a more detailed description here, which in any case would not be exhaustive, the skilled person being 25 able to select them on the basis of his general knowledge and according to the intended use or application and the desired organoleptic effect. In general terms, these perfuming co-ingredients belong to chemical classes as varied as alcohols, lactones, aldehydes, ketones, esters, ethers, acetates, nitriles, terpenoids, nitrogenous or sulphurous heterocyclic compounds and essential oils, and said perfuming co-ingredients can be of natural or synthetic origin. Many of these co-30 ingredients are in any case listed in reference texts such as the book by S. Arctander, Perfume and Flavor Chemicals, 1969, Montclair, New Jersey, USA, or its more recent versions, or in other works of a similar nature, as well as in the abundant patent literature in the field of perfumery. It is also understood that said co-ingredients may also be compounds known to release in a controlled manner various types of perfuming compounds (such as pro-perfumes). Non-limiting examples of suitable properfumes may include 4-(dodecylthio)-4-(2,6,6-trimethyl-2-cyclohexen-1-yl)-2-butanone, 4-(dodecylthio)-4-(2,6,6-trimethyl-1-cyclohexen-1-yl)-2-butanone, 3-(dodecylthio)-1-(2,6,6-trimethyl-3-cyclohexen-1-yl)-1-butanone, 2-(dodecylthio)octan-4-one, 2-phenylethyl oxo(phenyl)acetate, 3,7-dimethylocta-2,6-dien-1-yl oxo(phenyl)acetate, (Z)-hex-3-en-1-yl oxo(phenyl)acetate, 3,7-dimethyl-2,6-octadien-1-yl hexadecanoate, bis(3,7-dimethylocta-2,6-dien-1-yl) succinate, (2-((2-methylundec-1-en-1-yl)oxy)ethyl)benzene, 1-methoxy-4-(3-methyl-4-phenethoxybut-3-en-1-yl)benzene, (3-methyl-4-phenethoxybut-3-en-1-yl)benzene, 1-(((Z)-hex-3-en-1-yl)oxy)-2-methylundec-1-ene, (2-((2-methylundec-1-en-1-yl)oxy)ethoxy)benzene, 2-methyl-1-(octan-3-yloxy)undec-1-ene, 1-methoxy-4-(1-phenethoxyprop-1-en-2-yl)benzene, 1-methyl-4-(1-phenethoxyprop-1-en-2-yl)benzene, 2-(1-phenethoxyprop-1-en-2-yl)naphthalene, (2-phenethoxyvinyl)benzene, 2-(1-((3,7-dimethyloct-6-en-1-yl)oxy)prop-1-en-2-yl)naphthalene, (2-((2-pentylcyclopentylidene)methoxy)ethyl)benzene, 4-allyl-2-methoxy-1-((2-methoxy-2-phenylvinyl)oxy)benzene, (2-((2-heptylcyclopentylidene)methoxy)ethyl)benzene, 1-isopropyl-4-methyl-2-((2-pentylcyclopentylidene)methoxy)benzene, 2-methoxy-1-((2-pentylcyclopentylidene)methoxy)-4-propylbenzene, 3-methoxy-4-((2-methoxy-2-phenylvinyl)oxy)benzaldehyde, 4-((2-(hexyloxy)-2-phenylvinyl)oxy)-3-methoxybenzaldehyde or a mixture thereof.

By “perfumery adjuvant” it is herein understood an ingredient capable of imparting additional 5 added benefit such as a color, a particular light resistance, chemical stability, etc. A detailed description of the nature and type of adjuvant commonly used in perfuming bases cannot be exhaustive, but it has to be mentioned that said ingredients are well known to a person skilled in the art.

According to an embodiment, the core-shell microcapsule slurry or core-shell microcapsule of the invention (first type of delivery system) can be used in combination with a second type of delivery system, preferably microcapsule

Thus, according to a particular embodiment, the perfuming composition comprises:
the core-shell microcapsule slurry or core-shell microcapsule of the invention as a first type of delivery system, and
a second type of delivery system, wherein the first type of delivery system and the second type of delivery system differ in their perfuming formulations and/or carrier material (shell or matrix) and/or outer coating.

The core-shell microcapsule slurry or core-shell microcapsule of the present invention can advantageously be used in many application fields and used in perfumed consumer products.

The present invention also relates to a perfumed consumer product comprising
a personal care, home care or fabric care active base;
at least one core-shell microcapsule comprising
   an oil-based core comprising a hydrophobic material, preferably a perfume,
   a polymeric shell and
   a coating comprising a first deposition aid and
a second deposition aid,

wherein the first and second deposition aid are different deposition aids.

The embodiments and definitions for the oil-based core comprising a hydrophobic material, the polymeric shell, the first and second deposition aid as described herein-above applies mutatis mutandis.

In a particular embodiment, the perfumed consumer product is chosen in the group consisting of personal care composition, home care composition or fabric care composition, preferably in form of antiperspirants, hair care products, such as shampoo or hair-conditioner, body care products such as a shower gel, oral care products, laundry care products, preferably a detergent or a fabric softener.

Delivery systems can be used in liquid form applicable to liquid consumer products as well as in powder form, applicable to powder consumer products.

The consumer products of the invention, can in particular be of used in perfumed consumer products such as product belonging to fine fragrance or “functional” perfumery. Functional perfumery is chosen in the group consisting of personal care composition, home care composition or fabric care composition, preferably in form of antiperspirants, hair care products, such as shampoo or hair-conditioner, body care products such as a shower gel, oral care products, laundry care products, preferably a detergent or a fabric softener.

In particular a liquid consumer product comprising:
from 2 to 65% by weight, relative to the total weight of the consumer product, of at least one surfactant;
water or a water-miscible hydrophilic organic solvent; and
a perfuming composition or core-shell microcapsule slurry or core-shell microcapsule as described herein above.

Also a powder consumer product comprising
from 2 to 65% by weight, relative to the total weight of the consumer product, of at least one surfactant; and
a perfuming composition or core-shell microcapsule slurry or core-shell microcapsule as described herein above.

For the sake of clarity, it has to be mentioned that, by “perfumed consumer product” it is meant a consumer product which is expected to deliver among different benefits a perfuming effect to the surface to which it is applied (e.g. skin, hair, textile, paper, or home surface) or in the air (air-freshener, deodorizer etc). In other words, a perfumed consumer product according to the invention is a manufactured product which comprises a functional formulation also referred to as “base”, together with benefit agents, among which an effective amount of microcapsules according to the invention.

The nature and type of the other constituents of the perfumed consumer product do not warrant a more detailed description here, which in any case would not be exhaustive, the skilled person being able to select them on the basis of his general knowledge and according to the nature and the desired effect of said product. Base formulations of consumer products in which the microcapsules of the invention can be incorporated can be found in the abundant literature relative to such products. These formulations do not warrant a detailed description here which would in any case not be exhaustive. The person skilled in the art of formulating such consumer products is perfectly able to select the suitable components on the basis of his general knowledge and of the available literature.

Non-limiting examples of suitable perfumed consumer products can be a fine perfume, a splash or eau de perfume, a cologne, a shave or after-shave lotion, a liquid or solid detergent, a mono or multi chamber unit dose detergent, a fabric softener, a fabric refresher, liquid or solid scent-boosters (PEG/urea or salts), a dryer sheet, an ironing water, a paper, a bleach, a carpet cleaners, curtain-care products, a shampoo, a coloring preparation, a color care product, a hair shaping product, a dental care product, a disinfectant, an intimate care product, a hair spray, a hair conditioning product, a vanishing cream, a deodorant or antiperspirant, hair remover, tanning or sun product, nail products, skin cleansing, a makeup, a perfumed soap, shower or bath mousse, oil or gel, or a foot/hand care products, a hygiene product, an air freshener, a “ready to use” powdered air freshener, a mold remover, furnisher care, wipe, a dish detergent or hard-surface detergent, a leather care product, a car care product.

In a particular embodiment, the perfumed consumer product is a liquid or solid detergent, a fabric softener, liquid or solid scent-boosters (e.g. using PEG/urea or salts), a shampoo, a shower gel, a hair conditioning product (e.g. leave-on or rinse-off), a deodorant or antiperspirant.

Another object of the invention is a consumer product comprising:
a personal care active base, and
the core-shell microcapsule slurry or core-shell microcapsule as described herein above or the perfuming composition as defined above,
   wherein the consumer product is in the form of a personal care composition.

Personal care active base in which the delivery system of the invention can be incorporated can be found in the abundant literature relative to such products. These formulations do not warrant a detailed description here which would in any case not be exhaustive. The person skilled in the art of formulating such consumer products is perfectly able to select the suitable components on the basis of his general knowledge and of the available literature.

The personal care composition is preferably chosen in the group consisting of a hair-care product (e.g. a shampoo, hair conditioner, a colouring preparation or a hair spray), a cosmetic preparation (e.g. a vanishing cream, body lotion or a deodorant or antiperspirant), a skin-care product (e.g. a perfumed soap, shower or bath mousse, body wash, oil or gel, bath salts, or a hygiene product), oral care product (toothpaste or mouthwash composition) or a fine fragrance product (e.g. Eau de Toilette—EdT).

Another object of the invention is a consumer product comprising:
a home care or a fabric care active base, and
the core-shell microcapsule slurry or core-shell microcapsule as described herein above as defined above or the perfuming composition as defined above,
   wherein the consumer product is in the form of a home care or a fabric care composition.

Home care or fabric care bases in which the delivery system of the invention can be incorporated can be found in the abundant literature relative to such products. These formulations do not warrant a detailed description here which would in any case not be exhaustive. The person skilled in the art of formulating such consumer products is perfectly able to select the suitable components on the basis of his general knowledge and of the available literature.

The home or fabric care composition is preferably chosen in the group consisting fabric softener, liquid detergent, powder detergent, liquid scent booster and solid scent booster.

For liquid consumer product mentioned below, by “active base”, it should be understood that the active base includes active materials (typically including surfactants) and water.

For solid consumer product mention below, by “active base”, it should be understood that the active base includes active materials (typically including surfactants) and auxiliary agents (such as bleaching agents, buffering agent; builders; soil release or soil suspension polymers; granulated enzyme particles, corrosion inhibitors, antifoaming, sud suppressing agents; dyes, fillers, and mixtures thereof).

### Fabric Softener

An object of the invention is a consumer product in the form of a fabric softener composition comprising:
a fabric softener active base; preferably comprising at least one active material chosen in the group consisting of dialkyl quaternary ammonium salts, dialkyl ester quaternary ammonium salts (esterquats), Hamburg esterquat (HEQ), TEAQ (triethanolamine quat), silicones and mixtures thereof, the active base being used preferably in an amount comprised between 85 and 99.95% by weight based on the total weight of the composition,
a microcapsule slurry or microcapsules as defined above, preferably in an amount comprised between 0.05 to 15 wt %, more preferably between 0.1 and 5 wt % by weight based on the total weight of the composition,
optionally free perfume oil.

### Liquid Detergent

An object of the invention is a consumer product in the form of a liquid detergent composition comprising:
a liquid detergent active base; preferably comprising at least one active material chosen in the group consisting of anionic surfactant such as alkylbenzenesulfonate (ABS), secondary alkyl sulfonate (SAS), primary alcohol sulfate (PAS), lauryl ether sulfate (LES), methyl ester sulfonate (MES) and nonionic surfactant such as alkyl amines, alkanolamide, fatty alcohol poly(ethylene glycol) ether, fatty alcohol ethoxylate (FAE), ethylene oxide (EO) and propylene oxide (PO) copolymers, amine oxydes, alkyl polyglucosides, alkyl polyglucosamides, the active base being used preferably in an amount comprised between 85 and 99.95% by weight based on the total weight of the composition,
a microcapsule slurry or microcapsules as defined above, preferably in an amount comprised between 0.05 to 15 wt %, more preferably between 0.1 and 5 wt % by weight based on the total weight of the composition,
optionally free perfume oil.

### Solid Detergent

An object of the invention is a consumer product in the form of a solid detergent composition comprising:
a solid detergent active base; preferably comprising at least one active material chosen in the group consisting of anionic surfactant such as alkylbenzenesulfonate (ABS), secondary alkyl sulfonate (SAS), primary alcohol sulfate (PAS), lauryl ether sulfate (LES), methyl ester sulfonate (MES) and nonionic surfactant such as alkyl amines, alkanolamide, fatty alcohol poly(ethylene glycol) ether, fatty alcohol ethoxylate (FAE), ethylene oxide (EO) and propylene oxide (PO) copolymers, amine oxydes, alkyl polyglucosides, alkyl polyglucosamides, the active base being used preferably in an amount comprised between 85 and 99.95% by weight based on the total weight of the composition,
a microcapsule powder or microcapsule slurry or microcapsules as defined above, preferably in an amount comprised between 0.05 to 15 wt %, more preferably between 0.1 and 5 wt % by weight based on the total weight of the composition,
optionally free perfume oil.

### Shampoo/Shower Gel

An object of the invention is a consumer product in the form of a shampoo or a shower gel composition comprising:
a shampoo or a shower gel active base; preferably comprising at least one active material chosen in the group consisting of sodium alkylether sulfate, ammonium alkylether sulfates, alkylamphoacetate, cocamidopropyl betaine, cocamide MEA, alkylglucosides and amino acid based surfactants and mixtures thereof, the active base being used preferably in an amount comprised between 85 and 99.95% by weight based on the total weight of the composition,
a microcapsule slurry or microcapsules as defined above, preferably in an amount comprised between 0.05 to 15 wt %, more preferably between 0.1 and 5 wt % by weight based on the total weight of the composition,
optionally free perfume oil.

### Rinse-Off Conditioner

An object of the invention is a consumer product in the form of a rinse-off conditioner composition comprising:
a rinse-off conditioner active base; preferably comprising at least one active material chosen in the group consisting of cetyltrimonium chloride, stearyl trimonium chloride, benzalkonium chloride, behentrimonium chloride and mixture thereof, the active base being used preferably in an amount comprised between 85 and 99.95% by weight based on the total weight of the composition,
a microcapsule slurry or microcapsules as defined above, preferably in an amount comprised between 0.05 to 15 wt %, more preferably between 0.1 and 5 wt % by weight based on the total weight of the composition,
optionally free perfume oil.

### Solid Scent Booster

An object of the invention is a consumer product in the form of a solid scent booster composition comprising:
a solid carrier, preferably chosen in the group consisting of urea, sodium chloride, sodium sulphate, sodium acetate, zeolite, sodium carbonate, sodium bicarbonate, clay, talc, calcium carbonate, magnesium sulfate, gypsum, calcium sulfate, magnesium oxide, zinc oxide, titanium dioxide, calcium chloride, potassium chloride, magnesium chloride, zinc chloride, saccharides such as sucrose, mono-, di-, and polysaccharides and derivatives such as starch, cellulose, methyl cellulose, ethyl cellulose, propyl cellulose, polyols/sugar alcohols such as sorbitol, maltitol, xylitol, erythritol, and isomalt, PEG, PVP, citric acid or any water soluble solid acid, fatty alcohols or fatty acids and mixtures thereof,
a microcapsule slurry or microcapsules as defined above, in a powdered form, preferably in an amount comprised between 0.05 to 15 wt %, more preferably between 0.1 and 5 wt % by weight based on the total weight of the composition,
optionally free perfume oil.

### Liquid Scent Booster

An object of the invention is a consumer product in the form of a liquid scent booster composition comprising:
an aqueous phase,
a surfactant system essentially consisting of one or more than one non-ionic surfactant, wherein the surfactant system has a mean HLB between 10 and 14, preferably chosen in the group consisting of ethoxylated aliphatic alcohols, POE/PPG (polyoxyethylene and polyoxypropylene) ethers, mono and polyglyceryl esters, sucrose ester compounds, polyoxyethylene hydroxylesters, alkyl polyglucosides, amine oxides and combinations thereof;
a linker chosen in the group consisting of alcohols, salts and esters of carboxylic acids, salts and esters of hydroxyl carboxylic acids, fatty acids, fatty acid salts, glycerol fatty acids, surfactant having an HLB less than 10 and mixtures thereof, and
a microcapsule slurry or microcapsules as defined above, in the form of a slurry, preferably in an amount comprised between 0.05 to 15 wt %, more preferably between 0.1 and 5 wt % by weight based on the total weight of the composition,
optionally free perfume oil.

### Hair Coloration

An object of the invention is a consumer product in the form of an oxidative hair coloring composition comprising:
an oxidizing phase comprising an oxidizing agent and an alkaline phase comprising an alkakine agent, a dye precursor and a coupling compound; wherein said dye precursor and said coupling compound form an oxidative hair dye in the presence of the oxidizing agent, preferably in an amount comprised between 85 and 99.95% by weight based on the total weight of the composition,
microcapsules or microcapsule slurry as defined above, preferably in an amount comprised between 0.05 to 15 wt %, more preferably between 0.1 and 5 wt % by weight based on the total weight of the composition,
optionally free perfume oil

### Perfuming Composition

According to a particular embodiment, the consumer product is in the form of a perfuming composition comprising:
0.1 to 30%, preferably 0.1 to 20% of microcapsules or microcapsule slurry as defined previously,
0 to 40%, preferably 3-40% of perfume, and
20-90%, preferably 40-90% of ethanol, by weight based on the total weight of the perfuming composition.

The present invention also relates to a method for preparing a consumer product, wherein the method comprising the steps of
a) providing a personal care, home care or fabric care active base to form a mixture;
b) Adding a core-shell microcapsule slurry comprising at least one core-shell microcapsule to the mixture of step a), wherein the at least one core-shell microcapsule comprises
   an oil-based core comprising a hydrophobic material, preferably a perfume,
   a polymeric shell, and
   a coating comprising a first deposition aid,

wherein a second deposition aid is added in the active base of step a) or in the core-shell microcapsule slurry of step b).

According to a particular embodiment, the method comprises the steps of
a) adding the second deposition aid to a personal care, home care or fabric care active base to form a mixture;
b) Adding a core-shell microcapsule slurry comprising at least one core-shell microcapsule to the mixture of step a), wherein the at least one core-shell microcapsule comprises
   an oil-based core comprising a hydrophobic material, preferably a perfume,
   a polymeric shell and
   a coating comprising a first deposition aid.

The embodiments and definitions for the oil-based core comprising a hydrophobic material, the polymeric shell, the first and second deposition aid as described herein-above applies mutatis mutandis to the core-shell microcapsules per se. Furthermore, it is clear that the method of preparing a consumer product is in particular suitable for the preparation of the consumer product of the present invention.

According to the present invention, the second deposition aid is added to a personal care, home care or fabric care active base to form a mixture.

In a particular embodiment, the second deposition aid is added to the personal care, home care or fabric care active base in an amount of 0.01 to 10 wt. %, preferably 0.1 to 5 wt. %, more preferably 0.25 to 2.5 wt. %, based on the total weight of mixture in step a).

According to the present invention, the core shell microcapsule slurry comprising at least one core-shell microcapsule is added to the mixture in step a).

It is understood that any core-shell microcapsule slurry either commercially available or obtained from the preparation of the core-shell microcapsules can be applied.

In a particular embodiment, the mixture in step b) comprises the second deposition aid in an amount of 0.01 to 10 wt. %, preferably 0.1 to 5 wt. %, more preferably 0.25 to 2.5 wt. %, based on the total weight of the mixture of step b).

In a particular embodiment, the mixture is mixed at a temperature comprised between 5 and 90° C., preferably 10 to 80° C.

In a particular embodiment, the mixture is mixed for a time of 1 to 8 hours, preferably of 30 minutes to 5 hours.

The invention will now be further described by way of examples. It will be appreciated that the invention as claimed is not intended to be limited in any way by these examples

### EXAMPLES

### Example 1: Process and Composition

### Preparation of Capsules X

An aqueous solution of 10% wt. pork gelatine (A) is prepared separately.

A fragrance (Perfume A) to be encapsulated is mixed with poly-isocyanate (trimethylol propane-adduct of xylylene diisocyanate, Takenate® D-110N, Mitsui Chemical) (B).

Gum Arabic is dissolved in demineralised water to form the aqueous phase. The mixture is stirred until complete solubilisation and warmed at 40° C. Solution (B) is dispersed in the aqueous phase and emulsified by mechanical shear, static mixer, rotor-stator or rotor-rotor to obtain the desired particle size. Solution (A) is then added to the mixture under continued mechanical shear, the pH is adjusted to 4.45 using HCl 1M and maintained as such during 10 min.

Mechanical shear is maintained at the same rate and the solution is then subjected to a thermal treatment at 50-90° C. After a duration between 30 to 240 min, the mixture is cooled down to 10° C. at a controlled rate between 0.2 and 0.3° C.min⁻¹. The stirring speed is slightly decreased, and a cross-linking agent (glutaraldehyde aq. 50% wt. Supplied by Sigma-Aldrich) is finally added to the mixture. The capsule suspension is mixed during 4 to 10 hours at 20-25° C. to allow a complete reaction.

The result is an aqueous suspension or slurry of microcapsules.

TABLE 1
ingredients for capsule X

| Components | Wt. % |
|---|---|
| Water | 74.8 |
| Gum arabic¹⁾ | 1.1 |
| Gelatine²⁾ | 1.1 |
| Perfume³⁾ | 22.1 |
| | (Perfume A) |
| Takenate ®⁴⁾ | 0.40 |
| Lactic acid⁵⁾ | 0.5 |
| Glutaraldehyde⁶⁾ | 0.04 |
| Particle size (microns) | 72 |

¹⁾Nexira
²⁾PB Leiner
³⁾See table 2
⁴⁾ Trimethylol propane-adduct of xylylene diisocyanate, origin: Mitsui Chemicals, Inc., Japan, 75% solution of polyisocyanate in ethyl acetate
⁵⁾Purac Biochem, 90% aqueous solution
⁶⁾Sigma Aldrich, 50% aqueous solution

### Preparation of Capsules P

Gum Arabic (2.05 g) was dissolved in water (115.60 g). The solution was transferred into a reactor. In a round bottom flask, Uvinul A+ (4.28 g) and Takenate® D-110N (4.27 g) were dissolved in perfume oil B (85.48 g). Oil phase was dispersed in the aqueous solution with the help of Ultra-Turrax at 24,000 rpm for 2 min at room temperature. The resulting emulsion was warmed-up to 80° C. for 3 h to afford a white dispersion of microcapsules.

### Preparation of Capsules Y

Benzene-1,3,5-tricarbonyle chloride (1.73 g) was dissolved in benzyl benzoate (5 g). Sodium Caseinate (2 g) was dispersed in benzyl benzoate (5 g) and the dispersion was maintained under stirring at 60° C. for one hour. Both oil phases were mixed together, stirred at room temperature for 10 minutes, and then added to perfume oil A (25 g) at room temperature to form the oil phase. The latter was mixed with a solution of L-Lysine (2.53 g) in tap water (94.17 g). The reaction mixture was stirred with an Ultra Turrax at 24,000 rpm for 30 s to afford an emulsion. Ethylene diamine (0.12 g) and diethylene triamine (0.22 g) were dissolved in tap water (5 g) and this solution was added dropwise to the emulsion over the period of five minutes. The reaction mixture was stirred at 60° C. for 4 h to afford a white dispersion.

TABLE 2a
Formulation of the perfume oil A

| Ingredients | % in oil |
|---|---|
| Ethyl 2-methyl-pentanoate | 3.20% |
| Eucalyptol | 7.80% |
| 2,4-Dimethyl-3-cyclohexene-1-carbaldehyde | 0.75% |
| Aldehyde C10 | 0.75% |
| Citronellyl Nitrile | 4.30% |
| Isobornyl acetate | 3.00% |
| 2-tert-butyl-1-cyclohexyl acetate | 9.80% |
| Citronellyl Acetate | 1.30% |
| 2-Methylundecanal | 3.00% |
| Diphenyloxide | 0.80% |
| Aldehyde C12 | 1.30% |
| Dicyclopentadiene acetate | 9.85% |
| Ionone beta | 3.30% |
| Undecalactone gamma | 18.75% |
| Hexyl Salicylate | 15.90% |
| Benzyl Salicylate | 16.20% |

TABLE 2b
Formulation of the perfume oil B

| Raw material | qty (g) |
|---|---|
| carbinol acetate | 2.2 |
| citronellyl acetate | 16.59 |
| linalyl acetate | 10.72 |
| (−)-(1r)-2-(6,6-dimethyl-bicyclo[3.1.1]hept-2-en-2-yl)ethyl | 7.97 |
| acetate | |
| terpinyl acetate | 2.11 |
| verdyl acetate | 2.89 |
| Decanal | 0.07 |
| hexylcinnamic aldehyde | 13.94 |
| (+−)-ethyl 2-methylpentanoate | 0.26 |
| benzyl benzoate | 8.19 |
| allyl (cyclohexyloxy)acetate | 2.14 |
| cyclopentaneacetic acid, 3-oxo-2-pentyl-, methyl ester | 11.94 |
| hexyl isobutyrate | 2.63 |
| (2s)-2-{(2r)-2-[(1r)-4-methyl-3-cyclohexen-1- | 10.34 |
| yl]propyl}cyclopentanone (a) + | |
| (2r)-2-{(2s)-2-[(1r)-4-methyl-3-cyclohexen-1- | |
| yl]propyl}cyclopentanone (b) + | |
| (2s)-2-{(2s)-2-[(1r)-4-methyl-3-cyclohexen-1- | |
| yl]propyl}cyclopentanone (c) + | |
| (2r)-2-{(2r)-2-[(1r)-4-methyl-3-cyclohexen-1- | |
| yl]propyl}cyclopentanone (d) | |
| 1,3-Oxathiane, 2-methyl-4-propyl-, cis | 0.08 |
| Tetrahydro-4-methyl-2-(2-methylpropenyl)-2H-pyran | 0.44 |
| verdyl propionate | 4.33 |
| (3e)-4-(2,6,6-trimethyl-1-cyclohexen-1-yl)-3-buten-2-one | 0.52 |
| (3z)-3-hexen-1-yl(3z)-3-hexenoate | 1.24 |
| (1rs,2sr)-2,4-dimethyl-3-cyclohexene-1-carbaldehyde | 1.42 |

A cationic polymer was dissolved in water.
The cationic polymer aqueous solution was added to a capsules slurry P, X or Y to obtain a final loading of 1.5%, and the mixture kept under magnetic stirring at 60° C. for 1 h. The microcapsules are loaded with a UV-tracer (Uvinul A+)
Negatively charged biopolymer was dissolved in water to obtain a 5 wt % solution
The biopolymer solution was added to a rinse-off conditioner formulation (see composition below) to obtain a final loading of 0.5% and cationic microcapsules were added to the mixture at an equivalent oil loading of 0.3% or
The biopolymer solution was added to the cationic slurry at 0.5% and the final mixture was added to the rinse-off conditioner formulation at an equivalent oil loading of 0.3%.

### Alternatively:

A mixture of a cationic polymer and negatively charged biopolymer at a ratio of 2:1 and at 2% total polymer content is prepared
The mixture is added to a capsule slurry to obtain a final total polymer loading of 1.5%, and the mixture kept under magnetic stirring at 60° C. for 1 h.
The slurry is then added to a rinse-off conditioner formulation at an equivalent oil loading of 0.3%

TABLE 3
ingredients and amounts of the compositions

| | Effective Amount of Ingredient |
|---|---|
| Ingredients | (% wt)^{a)} |
| capsules slurry P, X or Y¹⁾ | 0.5-1% |
| rinse of conditioner | 90-98% |
| Chitosan Mw 1.8 MDa⁽²⁾ | 0.15-0.3% |
| Chitosan Mw 1.25 MDa⁽²⁾ | 0.15-0.3% |
| Modified Chitosan Mw 1.8 MDa⁽²⁾⁽⁴⁾ | 0.15-0.3% |
| Modified Chitosan Mw 1.25 MDa⁽²⁾⁽⁴⁾ | 0.15-0.3% |
| Chitosan Medium Mw⁽³⁾ | 0.15-0.3% |
| Pectin Citrus Fruit Aldrich⁽³⁾ | 0.05-05% |
| Carboxymethyl cellulose (CMC) | 0.05% |
| Alginate | 0.05% |

¹⁾see above
²⁾origin: Glentham Chemicals
³⁾origin: Sigma Aldrich
⁴⁾modification according to the following protocol:

Chitosan (5 g, Mw=1.8 MDa or 1.25 mDa) was dispersed in isopropanol (30 mL) in a 100 mL round bottom flask to afford a suspension. A solution of glycidyl trimethylammonium chloride in water (5 mL) was added at room temperature. The reaction mixture was stirred at 70° C. for 20 h and then cooled down to room temperature. The resulting suspension was poured into cold acetone and stored at 4° C. overnight. The copolymer as purified by dialysis at 1000 Da (Spectra/Por 7 membrane) and recovered by freeze-drying (conversion 20%).

TABLE 4
Rinse-off conditioner composition

| | | Concentration |
|---|---|---|
| | Ingredients | [wt %] |
| A | Water deionized | 81.8 |
| | Behentrimonium Chloride¹⁾ | 2.5 |
| | Hydroxyethylcellulose²⁾ | 1.5 |
| B | Cetearyl Alcohol³⁾ | 4 |
| | Glyceryl Stearate (and) PEG-100 Stearate⁴⁾ | 2 |
| | Behentrimonium Methosulfate (and) Cetyl alcohol | 4 |
| | (and) Butylene Glycol⁵⁾ | |
| | Ethoxy (20) Stearyl Alcohol⁶⁾ | 1 |
| C | Amodimethicone (and) Trideceth-12 (and) | 3 |
| | Cetrimonium Chloride⁷⁾ | |
| | Chlorhexidine Digluconate⁸⁾ 20% aqueous solution | 0.2 |
| D | Citric acid 10% aqueous sol. till pH 3.5-4 | q.s. |
| | TOTAL: | 100 |

¹⁾Genamin KDM P, Clariant
²⁾Tylose H10 Y G5, Shin Etsu
³⁾Lanette O, BASF
⁴⁾Arlacel 165-FP-MBAL-PA-(RB), Croda
⁵⁾Incroquat Behenyl TMS-50-MBAL-PA-(MH) HA4112, Croda
⁶⁾SP Brij S20 MBAL-PA(RB), Croda
⁷⁾Xiameter DC MEM-0949 Emulsion, Dow Corning
⁸⁾Alfa Aesar

### Example 2: Performance Measurement—Methods

### 2.1. Deposition of Microcapsules on Hair

The deposition enhancing properties of the polymers have been tested by measuring the amount of capsules deposited on 0.5 g mini-hair swatches from the rinse off conditioner formulation of the present invention.

1) CONTROLS: 0.1 mL of ROC (Rinse-off conditioner) formulation, is pipetted to a pre-weighed 20 mL scintillation vial using a 100 μL positive displacement and formulation mass recorded. The operation repeated 3 times.

2) SAMPLES: Wet a 500 mg mini brown Caucasian hair swatch with 40 mL of tap water (37-39° C.), 20 mL on each side, aimed at the vertically held hair mount with a large syringe. Gently squeeze out the excess water in a downward direction once. Apply 0.1 mL of ROC formulation evenly down the length of one side of the hair swatch with a 100 μL positive displacement pipet. Distribute the formulation with 10 rubs, massaging from top to bottom, followed by 10 gentle smoothing wipes using the thumb and pointer fingers of gloved hands. Rinse the swatch with 100 mL of tap water (37-39° C.) with 50 mL applied to each side of the swatch aimed at the hair mount. Gently squeeze out the excess water in a downward direction once. Finely cut the hair swatch (approximately 1 cm lengths) into a pre-weighed 20 mL scintillation vial. Repeat this process three times and then dry the uncapped vials containing the cut hair in a vacuum oven at 50-60° C. (˜80-100 Torr) for at least 5 hours (usually overnight). After the drying process, record the mass of the vials again to determine the mass of the hair.

3) EXTRACTION: Add 4 mL of 200 proof ethanol to each vial (3 controls and 3 cut/dried hair samples). Sonicate the vials for 60 min at room temperature. After sonication, filter the samples through a 0.45 μm, 25 mm PTFE syringe filter into a clean 4 dram vial. Dilute the control samples 10 fold in a 2 ml autosampler vial with 200 proof ethanol and DI water (650 μL EtOH, 250 μL DI water, and 100 μL control sample filtrate). Dilute the hair samples in a 2 ml autosampler vial with DI water only (250 μL DI water and 750 μL hair sample filtrate). Shake the diluted samples well and then analyze by HPLC using a UV detector.

### 2.2. Viscosity of the Rinse Off Conditioner Formulations

The viscosity of the ROC formulations has been measured by means of a rheometer TA Discovery HR-2, using the following method:
40 mm 2° cone-plate
Flowcurve@23° C.; soak time 30 s; 600 s log mode; initial shear rate 500 to 1 1/s; points per decade 5

### 2.3. Microscopy Survey of the Rinse Off Conditioner Formulations

The ROC formulations have been diluted 10× in DI water and imaged using an optical microscope at two different magnification (10× and 4×). The scope is to verify the presence of aggregates/cluster.

### 2.4. Characterization of Modified Microcapsules

The Z potential of the capsules before and after coating has been measured by means of a Malvern Zetasizer Nano, by diluting the slurry in a 1 mM NaCl solution. And the size measured by DLS (Malvern Mastersizer) and the presence of aggregates verified by optical microscopy.

### Example 3: Microcapsules Characterization: Cationic Coating and Cationic Coating+Anionic Coating

### 3.1. Characterization of the Microcapsules by Zeta Potential and Microscopy

TABLE 5
Formulation of different microcapsules and characterization

| | first | Second | | Size (light | Aggregates |
|---|---|---|---|---|---|
| capsule | coating | coating | Z (mV) | scattering) | in slurry |
| P | — | — | −40.1 | 9.9 | NO |
| P | Modified | — | 57.5 | 20 | NO |
| | chitosan 1.8 | | | | |
| | Mda + 20% Q | | | | |
| P | Modified | — | 68.5 | 28.8 | NO |
| | chitosan | | | | |
| | 1.25 | | | | |
| | Mda + 20% Q | | | | |
| P | Modified | CMC | 60.3 | | NO |
| | chitosan | | | | |
| | 1.25 | | | | |
| | Mda + 20% Q | | | | |
| P | Modified | Alginate | 62.9 | — | NO |
| | chitosan | | | | |
| | 1.25 | | | | |
| | Mda + 20% Q | | | | |
| P | Modified | Pectin | 63.9 | | NO |
| | chitosan | | | | |
| | 1.25 | | | | |
| | Mda + 20% Q | | | | |
| P | Chitosan | — | 61.7 | 10.62 | NO |
| | MMw (100- | | | | |
| | 310 kDa) | | | | |
| P | Chitosan | Pectin | 62 | | NO |
| | MMw (100- | | | | |
| | 310 kDa) | | | | |
| P | Salcare ® | — | 44.3 | 65.93 | YES |
| | SC60 | | | | |
| P | Salcare ® | CMC | −24.7 | 61.6 | N.D. |
| | SC60 | | | | |
| P | LR400 | Pectin | −13 | 36.5 | NO |
| P | Chitosan | — | 64.7 | | NO |
| | 1.8 Mw non | | | | |
| | modified + | | | | |
| | CMC | | | | |
| | mixture | | | | |
| X | — | — | −12 | 45.01 | N.D. |
| X | Chitosan | — | 67.2 | 55.5 | N.D. |
| | 1.8 Mw | | | | |
| | (non | | | | |
| | modified) | | | | |
| X | Chitosan | — | 40.3 | | NO |
| | 1.8 Mw non | | | | |
| | modified + | | | | |
| | CMC | | | | |
| | mixture | | | | |
| X | Chitosan | — | 47.5 | | NO |
| | MMw non | | | | |
| | modified | | | | |
| | (100-310 | | | | |
| | kDa) + | | | | |
| | Pectin | | | | |
| | mixture | | | | |
| Y | — | — | 27 | — | NO |
| Y | Chitosan | — | 65.4 | — | NO |
| | 1.8 Mw | | | | |
| | (non | | | | |
| | modified) | | | | |

N.D. - not determined

### 3.2. Deposition Tests for the Different Compositions

TABLE 6
Formulation of different microcapsules and results in the deposition tests

| | | Second | Second | Second | | | |
|---|---|---|---|---|---|---|---|
| | First | deposition | deposition | deposition | | | |
| | deposition | aid in | aid in | aid in | | | |
| Capsule | aid | coating | slurry | conditioner | Viscosity | Deposition | Type |
| P | — | — | — | — | — | 5 | Comp. |
| P | Modified | — | — | — | 2.3 | 13 | Comp. |
| | chitosan | | | | | | |
| | 1.8 + 20% Q | | | | | | |
| | Mda | | | | | | |
| P | Modified | — | — | Pectin - | — | 29.4 | Invent. |
| | chitosan | | | powder | | | |
| | 1.8 + 20% Q | | | | | | |
| | Mda | | | | | | |
| P | Modified | — | — | Pectin | 6.1 | 76.9 | Invent. |
| | chitosan | | | | | | |
| | 1.8 + 20% Q | | | | | | |
| | Mda | | | | | | |
| P | Modified | — | — | — | 2.4 | 22.9 | Comp. |
| | chitosan | | | | | | |
| | 1.25 + 20% Q | | | | | | |
| | Mda | | | | | | |
| P | Modified | — | — | Pectin | 8.5 | 57.1 | Invent. |
| | chitosan | | | | | | |
| | 1.25 + 20% Q | | | | | | |
| | Mda | | | | | | |
| P | Modified | — | Pectin | — | 1.8 | 44 | Invent. |
| | chitosan | | | | | | |
| | 1.25 + 20% Q | | | | | | |
| | Mda | | | | | | |
| P | Modified | — | — | CMC | 1.8 | 28.7 | Invent. |
| | chitosan | | | | | | |
| | 1.25 + 20% Q | | | | | | |
| | Mda | | | | | | |
| P | Modified | — | CMC | — | 1.7 | 32.4 | Invent. |
| | chitosan | | | | | | |
| | 1.25 + 20% Q | | | | | | |
| | Mda | | | | | | |
| P | Chitosan | — | — | — | 1.7 | 13.6 | Comp. |
| | MMw | | | | | | |
| P | Chitosan | — | — | Pectin | 3.6 | 61.5 | Invent. |
| | MMw | | | | | | |
| P | Chitosan | — | Pectin | — | 1.6 | 27.7 | Invent. |
| | MMw | | | | | | |
| P | Salcare ® | — | — | — | 3.12 | 26.2 | Comp. |
| | SC60 | | | | | | |
| P | Salcare ® | — | CMC | — | 2 | 37.6 | Invent. |
| | SC60 | | | | | | |
| P | — | — | Pectin | — | — | 23.6 | Comp. |
| P | — | — | — | Pectin | — | 6.1 | Comp. |
| P | Chitosan | CMC | — | — | — | 23.3 | Invent. |
| | 1.8 Mw non | mixture | | | | | |
| | modified | | | | | | |
| X | Chitosan | — | — | — | — | 7.5 | Comp. |
| | 1.8 mw | | | | | | |
| | (non | | | | | | |
| | modified) | | | | | | |
| X | Chitosan | — | — | Pectin | — | 37.6 | Invent. |
| | 1.8 mw | | | | | | |
| | (non | | | | | | |
| | modified) | | | | | | |
| X | Chitosan | CMC | — | — | — | 20.3 | Invent. |
| | 1.8 Mw non | mixture | | | | | |
| | modified | | | | | | |
| X | Chitosan | Pectin | — | — | — | 14 | Invent. |
| | MMw non | mixture | | | | | |
| | modified | | | | | | |
| | (100-310 | | | | | | |
| | kDa) | | | | | | |
| Y | Chitosan | — | — | — | — | 2.2 | Comp. |
| | 1.8 mw | | | | | | |
| | (non | | | | | | |
| | modified) | | | | | | |
| Y | Chitosan | — | — | Pectin | — | 24.6 | Invent. |
| | 1.8 mw | | | | | | |
| | (non | | | | | | |
| | modified) | | | | | | |

3.3. Conclusion from the Results

When the negatively charged polymer is added ta a microcapsule slurry comprising a positively charged coating, a significantly increased deposition can be obtained over the naked microcapsules or the microcapsules comprising the first deposition aid.

An improvement in deposition can be also observed for the cationic capsules when the negatively charged biopolymer is added directly into the final product (conditioner).

Additionally, an improvement in deposition can be observed when a pre-mixture of cationic and negatively charged biopolymer is added to the slurry, resulting in a single coating comprising both the cationic and negatively charged biopolymer, over the naked capsule or a capsule that only comprises the cationic deposition aid.

### Example 4: Fabric Conditioner Composition

Microcapsule slurry (see examples 2 and 3) are dispersed in a fabric conditioner base described in Table below to obtain a concentration of encapsulated perfume oil at 0.22%.

TABLE 7
Fabric conditioner composition

| Product | Origin | Wt % |
|---|---|---|
| Stepantex VL 90A | | 8.88 |
| Calcium Chloride Sol. 10% | | 0.36 |
| Proxel GXL | Avecia | 0.04 |
| Perfume | Firmenich SA | 1 |
| Water | | 89.72 |
| TOTAL | | 100 |

### Example 5: Liquid Detergent Composition

Microcapsule slurry (see examples 2 and 3) is dispersed in a liquid detergent base described in Table 8 to obtain a concentration of encapsulated perfume oil at 0.22%.

TABLE 8
Liquid detergent composition

| Ingredients | Concentration [wt %] |
|---|---|
| Sodium C14-17 Alkyl Sec Sulfonate¹⁾ | 7 |
| Fatty acids, C12-18 and C18-unsaturated²⁾ | 7.5 |
| C12/14 fatty alcohol polyglycol ether with | 17 |
| 7 mol EO³⁾ | |
| Triethanolamine | 7.5 |
| Propylene Glycol | 11 |
| Citric acid | 6.5 |
| Potassium Hydroxyde | 9.5 |
| Protease | 0.2 |
| Amylase | 0.2 |
| Mannanase | 0.2 |
| Acrylates/Steareth-20 Methacrylate | 6 |
| structuring Crosspolymer⁴⁾ | |
| Deionized Water | 27.4 |

¹⁾Hostapur SAS 60; Origin: Clariant
²⁾Edenor K 12-18; Origin: Cognis
³⁾Genapol LA 070; Origin: Clariant
⁴⁾Aculyn 88; Origin: Dow Chemical

### Example 6: Rinse-Off Conditioner

Microcapsule slurry (see examples 2 and 3) is dispersed in a rinse-off conditioner base described in table 9 to obtain a concentration of encapsulated perfume oil at 0.5%.

TABLE 9
Rinse-off conditioner composition

| | | Concentration |
|---|---|---|
| | Ingredients | [wt %] |
| A | Water deionized | 81.8 |
| | Behentrimonium Chloride¹⁾ | 2.5 |
| | Hydroxyethylcellulose²⁾ | 1.5 |
| B | Cetearyl Alcohol³⁾ | 4 |
| | Glyceryl Stearate (and) PEG-100 Stearate⁴⁾ | 2 |
| | Behentrimonium Methosulfate (and) Cetyl alcohol | 4 |
| | (and) Butylene Glycol⁵⁾ | |
| | Ethoxy (20) Stearyl Alcohol⁶⁾ | 1 |
| C | Amodimethicone (and) Trideceth-12 (and) | 3 |
| | Cetrimonium Chloride⁷⁾ | |
| | Chlorhexidine Digluconate⁸⁾ 20% aqueous solution | 0.2 |
| D | Citric acid 10% aqueous sol. till pH 3.5-4 | q.s. |
| | TOTAL: | 100 |

⁹⁾Genamin KDM P, Clariant
¹⁰⁾Tylose H10 Y G4, Shin Etsu
¹¹⁾Lanette O, BASF
¹²⁾Arlacel 165-FP-MBAL-PA-(RB), Croda
¹³⁾Incroquat Behenyl TMS-50-MBAL-PA-(MH) HA4112, Croda
¹⁴⁾SP Brij S20 MBAL-PA(RB), Croda
¹⁵⁾Xiameter DC MEM-0949 Emulsion, Dow Corning
¹⁶⁾Alfa Aesar

### Example 7: Shampoo Composition

Microcapsule slurry (see examples 2 and 3) is weighed and mixed in a shampoo composition to add the equivalent of 0.2% perfume.

TABLE 10
Shampoo composition

| | | Concentration |
|---|---|---|
| | Ingredients | [wt %] |
| A | Water deionized | 44.4 |
| | Polyquaternium-10¹⁾ | 0.3 |
| | Glycerin 85%²⁾ | 1 |
| | DMDM Hydantoin³⁾ | 0.2 |
| B | Sodium Laureth Sulfate⁴⁾ | 28 |
| | Cocamidopropyl Betaine⁵⁾ | 3.2 |
| | Disodium Cocoamphodiacetate⁶⁾ | 4 |
| | Ethoxy (20) Stearyl Alcohol⁶⁾ | 1 |
| C | Sodium Laureth Sulfate⁴⁾ | 3 |
| | Glyceryl Laureate⁷⁾ | 0.2 |
| D | Water deionized | 1 |
| | Sodium Methylparaben⁸⁾ | 0.1 |
| E | Sodium Chloride 10% aqueous sol. | 15 |
| | Citric acid 10% aqueous sol. till pH | q.s. |
| | 5.5-6 | |
| | Perfume | 0.5 |
| | TOTAL: | 100 |

¹⁾Ucare Polymer JR-400, Noveon
²⁾Schweizerhall
³⁾Glydant, Lonza
⁴⁾Texapon NSO IS, Cognis
⁵⁾Tego Betain F 50, Evonik
⁶⁾ Amphotensid GB 2009, Zschimmer & Schwarz
⁷⁾ Monomuls 90 L-12, Gruenau
⁸⁾ Nipagin Monosodium, NIPA

### Example 8: Antiperspirant Roll-on Emulsion Composition

Microcapsule slurry (see examples 2 and 3) is weighed and mixed in antiperspirant roll-on emulsion composition to add the equivalent of 0.2% perfume.

TABLE 11
Antiperspirant composition

| Ingredient | Amount (wt %) |
|---|---|
| Steareth-2¹⁾ (Part A) | 3.25 |
| Steareth-21²⁾ (Part A) | 0.75 |
| PPG-15 Stearyl Ether³⁾ (Part A) | 4 |
| WATER deionised (Part B) | 51 |
| Aluminum Chlorohydrate 50% | 40 |
| aqueous solution⁴⁾ (Part C) | |
| Fragrance (Part D) | 1 |

¹⁾BRIJ 72; origin: ICI
²⁾BRIJ 721; origin: ICI
³⁾ARLAMOL E; origin: UNIQEMA-CRODA
⁴⁾LOCRON L; origin: CLARIAN

Part A and B are heated separately to 75° C.; Part A is added to Part B under stirring and the mixture is homogenized for 10 min. Then, the mixture is cooled under stirring; and Part C is slowly added when the mixture reached 45° C. and Part D when the mixture reached at 35° C. while stirring. Then the mixture is cooled to room temperature.

### Example 9: Shower-Gel Composition

Microcapsule slurry (see examples 2 and 3) is weighed and mixed in the following composition to add the equivalent of 0.2% perfume.

TABLE 12
Shower gel composition

| Ingredients | Amount (% wt) |
|---|---|
| WATER deionised | 49.350 |
| Tetrasodium EDTA¹⁾ | 0.050 |
| Acrylates Copolymer²⁾ | 6.000 |
| Sodium C12-C15 Pareth Sulfate³⁾ | 35.000 |
| Sodium Hydroxide 20% aqueous solution | 1.000 |
| Cocamidopropyl Betaine⁴⁾ | 8.000 |
| Methylchloroisothiazolinone and Methylisothiazolinone⁵⁾ | 0.100 |
| Citric Acid (40%) | 0.500 |

⁵⁾EDETA B POWDER; trademark and origin: BASF
⁶⁾CARBOPOL AQUA SF-1 POLYMER; trademark and origin: NOVEON
⁷⁾ZETESOL AO 328 U; trademark and origin: ZSCHIMMER & SCHWARZ
⁸⁾TEGO-BETAIN F 50; trademark and origin: GOLDSCHMIDT
⁹⁾KATHON CG; trademark and origin: ROHM & HASS.

1. A core-shell microcapsule slurry comprising
at least one core-shell microcapsule, wherein the at least one core-shell microcapsule comprises
an oil-based core comprising a hydrophobic material,
a polymeric shell, and
a coating comprising a first deposition aid, and
a second deposition aid,
wherein the first and second deposition aids are different deposition aids.

2. The core-shell microcapsule slurry according to claim 1,
wherein the first and second deposition aids have opposite net charges at a pH when solubilized.

3. The core-shell microcapsule slurry according to claim 1, wherein the first deposition aid has a positive net charge at neutral or acidic pH.

4. The core-shell microcapsule slurry according to claim 1, wherein the first deposition aid comprises a biopolymer.

5. The core-shell microcapsule slurry according to claim 1, wherein the first deposition aid comprises a component selected from the group consisting of chitosan and a functionalized chitosan derivative.

6. The core-shell microcapsule slurry according to claim 5, wherein the chitosan or functionalized chitosan derivative has a molecular weight Mw from 3 kDa to 5 MDa.

7. The core-shell microcapsule slurry according to claim 1, wherein the first deposition aid is not crosslinked with the polymeric shell.

8. The core-shell microcapsule slurry according to claim 1, wherein the second deposition aid has a negative net charge at neutral or basic pH.

9. The core-shell microcapsule slurry according to claim 1, wherein the second deposition aid comprises a biopolymer.

10. The core-shell microcapsule slurry according to claim 1, wherein the second deposition aid comprises proteins, polysaccharides, and/or mixtures thereof.

11. The core-shell microcapsule slurry according to claim 1, wherein the second deposition aid comprises alginate, pectin, carboxymethyl-cellulose, whey protein, and/or gum arabic.

12. A perfuming composition comprising
a. a core-shell microcapsule slurry according to claim 1,
b. optionally an active ingredient,
c. at least one ingredient selected from the group consisting of a perfumery carrier and a perfumery base, and
d. optionally, at least one perfumery adjuvant.

13. A perfumed consumer product comprising
a personal care, home care and/or fabric care active base; and
at least one core-shell microcapsule comprising
an oil-based core comprising a hydrophobic material,
a polymeric shell, and
a coating comprising a first deposition aid and a second deposition aid,
wherein the first and second deposition aids are different deposition aids.

14. The perfumed consumer product according to claim 13, wherein the perfumed consumer product is selected from the group consisting of a personal care composition, a home care composition and a fabric care composition.

15. A method of preparing a consumer product, the method comprising the steps of
a. providing a personal care, home care, and/or fabric care active base to form a mixture; and
b. adding a core-shell microcapsule slurry comprising at least one core-shell microcapsule to the mixture of step a), wherein the at least one core-shell microcapsule comprises
an oil-based core comprising a hydrophobic material,
a polymeric shell, and
a coating comprising a first deposition aid,
wherein a second deposition aid is added in the active base of step a) or in the core shell microcapsule slurry of step b).

16. The core-shell microcapsule slurry according to claim 1, wherein the hydrophobic material is a perfume.

17. The core-shell microcapsule slurry according to claim 5, wherein the chitosan or functionalized chitosan derivative has a molecular weight Mw from 900 kDa to 4 MDa.

18. The core-shell microcapsule slurry according to claim 5, wherein the chitosan or functionalized chitosan derivative has a molecular weight Mw from 1 MDa to 3.5 MDa.

19. The core-shell microcapsule slurry according to claim 5, wherein the chitosan or functionalized chitosan derivative has a molecular weight Mw from 1.25 MDa to 1.8 MDa.

20. The perfumed consumer product according to claim 13, wherein the hydrophobic material is a perfume.
